(19) United States
(12) Patent Application Publication
(10) Pub. No.: US 2026/0034166 A1
(43) Pub. Date: Feb. 5, 2026

(54) HEPARIN COMPOSITIONS FOR TREATMENT OF LUNG DAMAGE AND METHODS OF USE THEREOF

(71) Applicant: Atossa Therapeutics, Inc., Seattle, WA (US)

(72) Inventor: Steven C. QUAY, Seattle, WA (US)

(21) Appl. No.: 18/995,011

(22) PCT Filed: Jul. 27, 2023
(86) PCT No.: PCT/US2023/028889

§ 371 Date: Jan. 15, 2025

Related U.S. Application Data
(60) Provisional application No. 63/369,741, filed on Jul. 28, 2022.

(51) Int. Cl. A61K 31/727; A61K 31/198; A61P 11/00; A61P 29/00; A61P 35/00; A61P 39/00

(52) U.S. Cl. CPC A61K 31/727; A61K 31/198; A61P 11/00; A61P 29/00; A61P 35/00; A61P 39/00

(57) ABSTRACT

Described herein are compositions and methods for treating lung damage associated with cancer therapy, for example radiation-induced lung damage or chemotherapy-induced lung damage. Lung damage may be treated by administering heparin, optionally formulated with or administered concurrently with N-acetylcysteine. The compositions may be nebulized and administered via inhalation to treat lung damage.

### CROSS-REFERENCE

This application claims the benefit of U.S. Provisional Application No. 63/369,741, entitled “HEPARIN COMPOSITIONS FOR TREATMENT OF LUNG DAMAGE AND METHODS OF USE THEREOF,” filed Jul. 28, 2022, which application is incorporated herein by reference.

### BACKGROUND

Chemoradiation therapies used for cancer treatment are associated with a wide range of serious side effects, including pulmonary damage. There is a need for therapies to treat or reduce pulmonary damage associated with chemoradiation therapies that may be administered in conjunction with cancer therapies.

### SUMMARY

In various aspects, the present disclosure provides a method of treating a radiation-induced lung damage in a subject in need thereof, the method comprising, administering to the subject via inhalation a nebulized composition comprising a heparin, thereby treating the radiation-induced lung damage in the subject.

In some aspects, the radiation-induced lung damage is caused by radiation therapy.

In various aspects, the present disclosure provides a method of treating a chemotherapy-induced lung damage in a subject in need thereof, the method comprising, administering to the subject via inhalation a nebulized composition comprising, a heparin, thereby treating the chemotherapy-induced lung damage in the subject.

In some aspects, the chemotherapy-induced lung damage is caused by administering a chemotherapy comprising bleomycin, carmustine, lomustine, busulfan, daunorubicin, doxorubicin, idarubicin, or combinations thereof.

In various aspects, the present disclosure provides a method of treating a lung damage, the method comprising administering via inhalation a nebulized composition comprising a heparin to a subject who has previously undergone cancer treatment, thereby treating the lung damage in the subject.

In some aspects, the subject has previously undergone treatment for cancer within the last six months.

In some aspects, the heparin comprises a low molecular weight heparin. In some aspects, the low molecular weight heparin comprises an average molecular weight of not less than 3 kilodaltons and not more than 8 kilodaltons. In some aspects, the low molecular weight heparin comprises an average molecular weight of not less than 3 kilodaltons and not more than 4 kilodaltons. In some aspects, the low molecular weight heparin comprises an average molecular weight of not less than 4 kilodaltons and not more than 5 kilodaltons. In some aspects, the low molecular weight heparin comprises an average molecular weight of not less than 5 kilodaltons and not more than 6 kilodaltons. In some aspects, the low molecular weight heparin comprises an average molecular weight of not less than 6 kilodaltons and not more than 7 kilodaltons.

In some aspects, the low molecular weight heparin comprises enoxaparin. In some aspects, the low molecular weight heparin comprises bemiparin, nadroparin, reviparin, parnaparin, certoparin, dalteparin, tinzaparin, or a combination thereof.

In some aspects, the composition comprises not less than 10 mg/mL and not more than 500 mg/mL of the heparin. In some aspects, the composition comprises not less than 25 mg/mL and not more than 250 mg/mL of the heparin. In some aspects, the composition comprises not less than 50 mg/mL and not more than 200 mg/mL of the heparin. In some aspects, the composition comprises about 100 mg/mL of the heparin.

In some aspects, the composition is aqueous. In some aspects, the method further comprises administering N-acetylcysteine to the subject via inhalation. In some aspects, the N-acetylcysteine is administered at a dose of not less than 100 mg and not more than 2000 mg per dose, or not less than 300 mg or not more than 800 mg per dose. In some aspects, the N-acetylcysteine is administered within about 1 hour of administering the composition. In some aspects, the composition comprises the N-acetylcysteine.

In some aspects, the subject has cancer. In some aspects, the subject is undergoing treatment for cancer. In some aspects, the treatment for the cancer comprises radiation therapy, chemotherapy, or a combination thereof. In some aspects, the cancer comprises a thoracic cancer. In some aspects, the thoracic cancer comprises lung cancer, thymic cancer, esophageal cancer, or tracheal cancer. In some aspects, the lung cancer is non-small cell lung cancer. In some aspects, the method further comprises slowing progression of the cancer in the subject.

In some aspects, the subject has pneumonitis. In some aspects, the subject has pulmonary fibrosis.

In some aspects, the method further comprises reducing a level of an inflammatory cytokine. In some aspects, the inflammatory cytokine comprises interleukin-6 (IL-6), interleukin-3 (IL-3), interleukin-7 (IL-7), interleukin-10 (IL-10), interleukin-1 (IL-1), angiopoietin-2, INF-γ, TNF-α, TGF-β, or combinations thereof.

In some aspects, the method further comprises reducing airway inflammation in the subject. In some aspects, the method further comprises reducing pulmonary fibrosis in the subject.

In some aspects, the method comprises administering the composition two times per day. In some aspects, the method comprises administering the composition for 14 days. In some aspects, the method comprises administering not less than 0.5 mg per kg patient weight (mg/kg) and not more than 2 mg/kg of the heparin per dose. In some aspects, the method comprises administering not less than 1 mg/kg and not more than 4 mg/kg of the heparin per day.

In some aspects, the composition comprises not more than 25 μg gold per gram of heparin, not more than 250 μg molybdenum per gram of heparin, not more than 75 μg chromium per gram of heparin, or combinations thereof. In some aspects, the composition comprises not more than 25 μg gold per gram of heparin, not more than 250 μg molybdenum per gram of heparin, and not more than 75 μg chromium per gram of heparin. In some aspects, the composition comprises not more than 2.5 μg gold per gram of heparin, not more than 25 μg molybdenum per gram of heparin, not more than 7.5 μg chromium per gram of heparin, or combinations thereof. In some aspects, the composition comprises not more than 2.5 μg gold per gram of heparin, not more than 25 μg molybdenum per gram of heparin, and not more than 7.5 μg chromium per gram of heparin.

In some aspects, the nebulized composition comprises an average droplet diameter of 1 μm to 12 μm. In some aspects, the nebulized composition comprises an average droplet diameter of 1 μm to 10 μm. In some aspects, the nebulized composition comprises an average droplet diameter of 1 μm to 8 μm. In some aspects, the nebulized composition comprises an average droplet diameter of 1 μm to 5 μm. In some aspects, the nebulized composition comprises an average droplet diameter of 2 μm to 4 μm. In some aspects, the nebulized composition comprises an average droplet diameter of 2 μm to 3 μm.

In various aspects, the present disclosure provides a composition comprising a heparin and not more than 25 μg gold per gram of heparin, not more than 250 μg molybdenum per gram of heparin, not more than 75 μg chromium per gram of heparin, or combinations thereof; wherein the composition is formulated for inhalation.

In some aspects, the heparin comprises a low molecular weight heparin. In some aspects, the low molecular weight heparin comprises enoxaparin. In some aspects, the low molecular weight heparin comprises bemiparin, nadroparin, reviparin, parnaparin, certoparin, dalteparin, tinzaparin, or a combination thereof.

In some aspects, the composition comprises not less than 10 mg/mL and not more than 500 mg/mL of the heparin. In some aspects, the composition comprises not less than 25 mg/mL and not more than 250 mg/mL of the heparin. In some aspects, the composition comprises not less than 50 mg/mL and not more than 200 mg/mL of the heparin. In some aspects, the composition comprises about 100 mg/mL of the heparin.

In some aspects, the composition is aqueous. In some aspects, the composition comprises further comprising N-acetylcysteine. In some aspects, the composition comprises no less than 1% and no more than 20%, no less than 1% and no more than 10%, no less than 2% and no more than 10%, or no less than 2% and no more than 5% (w/v)N-acetylcysteine.

In some aspects, the composition comprises not more than 25 μg gold per gram of heparin, not more than 250 μg molybdenum per gram of heparin, and not more than 75 μg chromium per gram of heparin. In some aspects, the composition comprises not more than 2.5 μg gold per gram of heparin, not more than 25 μg molybdenum per gram of heparin, not more than 7.5 μg chromium per gram of heparin, or combinations thereof. In some aspects, the composition comprises not more than 2.5 μg gold per gram of heparin, not more than 25 μg molybdenum per gram of heparin, and not more than 7.5 μg chromium per gram of heparin.

In some aspects, the composition comprises not less than 0.01 and not more than 7.5 μg gold per gram of heparin, not less than 0.01 and not more than 5 μg gold per gram of heparin, not less than 0.01 and not more than 2.5 μg gold per gram of heparin, not less than 0.01 and not more than 1 μg gold per gram of heparin, or not less than 0.001 and not more than 0.5 μg gold per gram of heparin.

In some aspects, the composition comprises not less than 1 and not more than 250 μg molybdenum per gram of heparin, not less than 1 and not more than 150 μg molybdenum per gram of heparin, not less than 1 and not more than 100 μg molybdenum per gram of heparin, not less than 0.1 and not more than 25 μg molybdenum per gram of heparin, or not less than 0.001 and not more than 0.5 μg molybdenum per gram of heparin.

In some aspects, the composition comprises not less than 0.1 and not more than 75 μg chromium per gram of heparin, not less than 0.1 and not more than 25 μg chromium per gram of heparin, not less than 0.1 and not more than 10 μg chromium per gram of heparin, not less than 0.01 and not more than 7.5 μg chromium per gram of heparin, or not less than 0.001 and not more than 0.5 μg chromium per gram of heparin.

In some aspects, the composition comprises not more than 5 μg arsenic per gram of heparin, not less than 0.01 and not more than 5 μg arsenic per gram of heparin, not less than 0.01 and not more than 4 μg arsenic per gram of heparin, not less than 0.01 and not more than 2.5 μg arsenic per gram of heparin, or not less than 0.001 and not more than 0.5 μg arsenic per gram of heparin.

In some aspects, the composition comprises not more than 2.5 μg mercury per gram of heparin, not less than 0.01 and not more than 2.5 μg mercury per gram of heparin, not less than 0.01 and not more than 1.5 μg mercury per gram of heparin, not less than 0.01 and not more than 0.5 μg mercury per gram of heparin, or not less than 0.001 and not more than 0.5 μg mercury per gram of heparin.

In some aspects, the composition comprises not more than 7.5 μg cobalt per gram of heparin, not less than 0.01 and not more than 7.5 μg cobalt per gram of heparin, not less than 0.01 and not more than 5 μg cobalt per gram of heparin, not less than 0.01 and not more than 2.5 μg cobalt per gram of heparin, or not less than 0.001 and not more than 0.5 μg cobalt per gram of heparin.

In some aspects, the composition comprises not more than 2.5 μg vanadium per gram of heparin, not less than 0.01 and not more than 2.5 μg vanadium per gram of heparin, not less than 0.01 and not more than 1.5 μg vanadium per gram of heparin, not less than 0.01 and not more than 0.5 μg vanadium per gram of heparin, or not less than 0.001 and not more than 0.5 μg vanadium per gram of heparin.

In some aspects, the composition comprises not more than 12.5 μg nickel per gram of heparin, not less than 0.1 and not more than 12.5 μg nickel per gram of heparin, not less than 0.01 and not more than 7.5 μg nickel per gram of heparin, not less than 0.01 and not more than 5 μg nickel per gram of heparin, or not less than 0.001 and not more than 0.5 μg nickel per gram of heparin.

In some aspects, the composition comprises not more than 2.5 μg palladium per gram of heparin, not less than 0.01 and not more than 2.5 μg palladium per gram of heparin, not less than 0.01 and not more than 1.5 μg palladium per gram of heparin, not less than 0.01 and not more than 0.5 μg palladium per gram of heparin, or not less than 0.001 and not more than 0.5 μg palladium per gram of heparin.

In some aspects, the composition comprises not more than 2.5 μg iridium per gram of heparin, not less than 0.01 and not more than 2.5 μg iridium per gram of heparin, not less than 0.01 and not more than 1.5 μg iridium per gram of heparin, not less than 0.01 and not more than 0.5 μg iridium per gram of heparin, or not less than 0.001 and not more than 0.5 μg iridium per gram of heparin.

In some aspects, the composition comprises not more than 2.5 μg osmium per gram of heparin, not less than 0.01 and not more than 2.5 μg osmium per gram of heparin, not less than 0.01 and not more than 1.5 μg osmium per gram of heparin, not less than 0.01 and not more than 0.5 μg osmium per gram of heparin, or not less than 0.001 and not more than 0.5 μg osmium per gram of heparin.

In some aspects, the composition comprises not more than 2.5 μg rhodium per gram of heparin, not less than 0.01 and not more than 2.5 μg rhodium per gram of heparin, not less than 0.01 and not more than 1.5 μg rhodium per gram of heparin, not less than 0.01 and not more than 0.5 μg rhodium per gram of heparin, or not less than 0.001 and not more than 0.5 μg rhodium per gram of heparin.

In some aspects, the composition comprises not more than 2.5 μg ruthenium per gram of heparin, not less than 0.01 and not more than 2.5 μg ruthenium per gram of heparin, not less than 0.01 and not more than 1.5 μg ruthenium per gram of heparin, not less than 0.01 and not more than 0.5 μg ruthenium per gram of heparin, or not less than 0.001 and not more than 0.5 μg ruthenium per gram of heparin.

In some aspects, the composition comprises not more than 17.5 μg silver per gram of heparin, not less than 0.1 and not more than 17.5 μg silver per gram of heparin, not less than 0.1 and not more than 10 μg silver per gram of heparin, not less than 0.01 and not more than 5 μg silver per gram of heparin, or not less than 0.001 and not more than 0.5 μg silver per gram of heparin.

In some aspects, the composition comprises not more than 2.5 μg platinum per gram of heparin, not less than 0.01 and not more than 2.5 μg platinum per gram of heparin, not less than 0.01 and not more than 1.5 μg platinum per gram of heparin, not less than 0.01 and not more than 0.5 μg platinum per gram of heparin, or not less than 0.001 and not more than 0.5 μg platinum per gram of heparin.

In some aspects, the composition comprises not more than 17.5 μg lithium per gram of heparin, not less than 0.1 and not more than 17.5 μg lithium per gram of heparin, not less than 0.1 and not more than 10 μg lithium per gram of heparin, not less than 0.01 and not more than 5 μg lithium per gram of heparin, or not less than 0.001 and not more than 0.5 μg lithium per gram of heparin.

In some aspects, the composition comprises not more than 50 μg antimony per gram of heparin, not less than 1 and not more than 50 μg antimony per gram of heparin, not less than 0.1 and not more than 25 μg antimony per gram of heparin, not less than 0.1 and not more than 10 μg antimony per gram of heparin, or not less than 0.001 and not more than 0.5 μg antimony per gram of heparin.

In some aspects, the composition comprises not more than 750 μg barium per gram of heparin, not less than 10 and not more than 750 μg barium per gram of heparin, not less than 10 and not more than 500 μg barium per gram of heparin, not less than 10 and not more than 250 μg barium per gram of heparin, or not less than 0.001 and not more than 0.5 μg barium per gram of heparin.

In some aspects, the composition comprises not more than 75 μg copper per gram of heparin, not less than 1 and not more than 75 μg copper per gram of heparin, not less than 0.1 and not more than 50 μg copper per gram of heparin, not less than 0.1 and not more than 25 μg copper per gram of heparin, or not less than 0.001 and not more than 0.5 μg copper per gram of heparin.

In some aspects, the composition comprises not more than 150 μg tin per gram of heparin, not less than 1 and not more than 150 μg tin per gram of heparin, not less than 0.1 and not more than 75 μg tin per gram of heparin, not less than 0.1 and not more than 50 μg tin per gram of heparin, or not less than 0.001 and not more than 0.5 μg tin per gram of heparin.

In some aspects, the composition comprises an average droplet diameter of 1 μm to 12 μm. In some aspects, the composition comprises an average droplet diameter of 1 μm to 10 μm. In some aspects, the composition comprises an average droplet diameter of 1 μm to 8 μm. In some aspects, the composition comprises an average droplet diameter of 1 μm to 5 μm. In some aspects, the composition comprises an average droplet diameter of 2 μm to 4 μm. In some aspects, the composition comprises an average droplet diameter of 2 μm to 3 μm.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

Described herein compositions comprising heparin to treat lung damage associated with chemoradiation therapy. A number of cancer treatments, including radiation therapy or chemotherapy with drugs such as bleomycin, carmustine, lomustine, busulfan, daunorubicin, doxorubicin, or idarubicin, are associated with lung damage. Chemoradiation-induced lung damage may include inflammation of the lungs (e.g., radiation-induced pneumonitis), fibrin deposition, fluid build-up, lung infection, secondary infection, pulmonary fibrosis, coagulation in the lungs, pulmonary venous thromboembolism, and other serious respiratory side effects. The heparin compositions described herein may be administered to a subject undergoing or who has previously undergone chemotherapy or radiation therapy to treat (e.g., reverse, prevent, or slow progression of) lung damage associated with the chemotherapy or radiation therapy. In some embodiments, the patient may be undergoing chemotherapy or radiation therapy for treatment of a cancer, such lung cancer (e.g., small cell lung cancer or non-small cell lung cancer) or other thoracic cancers (e.g., thymic cancer, esophageal cancer, or tracheal cancer). The heparin compositions described herein may be nebulized and administered via inhalation to directly target damaged lung tissues.

Heparin (e.g., nebulized enoxaparin) may treat chemotherapy-induced or radiation-induced lung damage by inhibiting the release of inflammatory cytokines (e.g., interleukin-6 (IL-6), interleukin-3 (IL-3), interleukin-7 (IL-7), interleukin-10 (IL-10), interleukin-1 (IL-1), angiopoietin-2, INF-γ, TNF-α, TGF-β, or combinations thereof) that can lead to treatment failure due to excessive inflammatory lung damage. In addition to treating lung damage associated with the chemotherapy or radiation therapy, heparin compositions (e.g., nebulized enoxaparina compositions) may also slow progression of the cancer in the subject, for example by inhibiting mutagenic proliferation, adhesion, angiogenesis, migration, or invasion of cancer cells. Anti-metastatic effects may involve inhibition of P-/L-selectin binding, angiogenesis, and interference with the CXCL12-CXCR4 axis. Administration of heparin to cancer patients (e.g., lung cancer patients) undergoing chemotherapy and/or radiation therapy may increase patient survival. For example, heparin therapies may prevent coagulation activation and pulmonary venous thromboembolism (VTE), which are hallmarks of malignant disease and represent a major cause of morbidity and mortality in patients with advanced cancer.

The lung is one of the most sensitive tissues to ionizing radiation, and its susceptibility to radiation damage limits the success of radiotherapy for lung cancer treatment, which in turn can shorten overall survival. In the early stage of Radiation Induced Lung Damage (RILI), damage-associated molecular pattern molecules (DAMPs) are released from cells to recruit many immune effector cells to accumulate the damage of lung tissue and contribute to tissue remodeling. Under the induction of intercellular cell adhesion molecule-1 (ICAM-1) and platelet endothelial cell adhesion molecule-1 (PECAM-1/CD31), neutrophils and macrophages arrive one after another and release IL-3, IL-6, IL-7, TNF-α, and TGF-β to produce an inflammatory reaction. In the late stage, T helper 2 (Th2) cells participate in the profibrotic process. Neutrophils and macrophages induced profibrotic effects via the secretion of TGF-β, IL-6, and PDGF. Blood monocytes are recruited to lungs and differentiate into fibroblasts and myofibroblasts. Neutrophils secrete elastase and matrix metalloproteinases to contribution to accumulation ECM. Finally, irreversible pulmonary fibrosis occurs. Radiation-induced lung damage affects 30-40% of lung cancer patients, and about 35% of esophageal cancer patients. In non-small cell cancer patients receiving concurrent chemotherapy and radiation therapy the incidence of radiation-induced lung damage is estimated to be greater than 60%.

Heparin therapies (e.g., inhaled enoxaparin) targets the overactive cytokine response to combat radiation induced lung damage without interfering with standard of care cancer therapy. IH Enoxaparin is such a therapy. Inhaled heparin provides a targeted-drug delivery therapy that acts directly on the lung parenchyma, but also acts as an anti-inflammatory by inducing a reduction in the release of interleukin (IL)-6 and other inflammatory cytokines.

Nebulized administration of heparin (e.g., cnoxaparin) provides localized high drug concentrations in the epithelium of the airway, with limited systemic absorption. Heparin delivery via inhalation is therefore simple and safe to administer by ventilator nebulizer without compromising standard of care therapy. The lack of systemic exposure allows for a targeted-drug therapy without the risk of drug-drug interactions of systemic side effects that could potentially interfere with concurrent chemoradiotherapy.

A heparin composition described herein may further comprise one or more additional active agents. For example, a heparin composition may further comprise N-acetylcysteine. Alternatively, or in addition, a heparin composition may be administered in combination with an additional composition comprising an active agent. For example, a heparin composition may be administered in combination with a composition comprising N-acetylcysteine.

### Compositions for Treatment of Lung Damage

A composition of the present disclosure may comprise one or more active agents. In some embodiments, an active agent may be an agent to treat pulmonary damage (e.g., pulmonary damage associated with radiation therapy, chemotherapy, or both). In some embodiments, an active agent may be a polyanionic electrolyte (e.g., poly-glutamate, poly-aspartate, alginate, carboxy-methyl-cellulose, polyacrylic acid, keratin sulfate, heparan sulfate, or heparin). For example, an active agent may be a low molecular weight heparin (e.g., enoxaparin, bemiparin, nadroparin, reviparin, parnaparin, certoparin, dalteparin, tinzaparin, or a combination thereof). In some embodiments, a composition may further comprise an antioxidant (e.g., N-acetylcysteine). A composition to treat lung damage may comprise heparin. A composition to treat lung damage may comprise heparin and N-acetylcysteine.

In some embodiments, the heparin may comprise a molecular weight of from 5 kilodaltons (kDa) to 40 kDa (5000 g/mol to 40,000 g/mol). In some embodiments, the heparin may be a low molecular weight heparin, such as enoxaparin, bemiparin, nadroparin, reviparin, parnaparin, certoparin, dalteparin, tinzaparin, or a combination thereof. The low molecular weight heparin may comprise an average molecular weight of less than 8 kDa (8000 g/mol). In some embodiments, the low molecular weight heparin may comprise an average molecular weight of from 1 kDa to 8 kDa (1000 g/mol to 8000 g/mol), from 1.5 kDa to 8 kDa (1500 g/mol to 8000 g/mol), 2 kDa to 8 kDa (2000 g/mol to 8000 g/mol), 2.5 kDa to 8 kDa (2500 g/mol to 8000 g/mol), 3 kDa to 8 kDa (3000 g/mol to 8000 g/mol), 3.5 kDa to 8 kDa (3500 g/mol to 8000 g/mol), 4 kDa to 8 kDa (4000 g/mol to 8000 g/mol), 1 kDa to 6 kDa (1000 g/mol to 6000 g/mol), from 1.5 kDa to 6 kDa (1500 g/mol to 6000 g/mol), 2 kDa to 6 kDa (2000 g/mol to 6000 g/mol), 2.5 kDa to 6 kDa (2500 g/mol to 6000 g/mol), 3 kDa to 6 kDa (3000 g/mol to 6000 g/mol), 3.5 kDa to 6 kDa (3500 g/mol to 6000 g/mol), 4 kDa to 6 kDa (4000 g/mol to 6000 g/mol), 1 kDa to 5 kDa (1000 g/mol to 5000 g/mol), from 1.5 kDa to 5 kDa (1500 g/mol to 5000 g/mol), 2 kDa to 5 kDa (2000 g/mol to 5000 g/mol), 2.5 kDa to 5 kDa (2500 g/mol to 5000 g/mol), 3 kDa to 5 kDa (3000 g/mol to 5000 g/mol), 3.5 kDa to 5 kDa (3500 g/mol to 5000 g/mol), or 4 kDa to 5 kDa (4000 g/mol to 5000 g/mol). For example, the low molecular weight heparin may comprise an average molecular weight of 3 kDa to 7 kDa (3000 g/mol to 7000 g/mol). In another example, the low molecular weight heparin may comprise an average molecular weight of 3 kDa to 4 kDa (3000 g/mol to 4000 g/mol). In another example, the low molecular weight heparin may comprise an average molecular weight of 4 kDa to 5 kDa (4000 g/mol to 5000 g/mol). In another example, the low molecular weight heparin may comprise an average molecular weight of 5 kDa to 6 kDa (5000 g/mol to 6000 g/mol). In another example, the low molecular weight heparin may comprise an average molecular weight of 6 kDa to 7 kDa (6000 g/mol to 7000 g/mol).

A method of the present disclosure may comprise administering a composition comprising an active agent (e.g., a composition comprising heparin) to a subject in need thereof via inhalation. The composition may be formulated for delivery via inhalation. A method of treating pulmonary damage (e.g., pulmonary damage associated with radiation therapy, chemotherapy, or both) may comprise delivering a composition of the present disclosure to the lungs of a subject in need thereof by administering the composition by inhalation. In some embodiments, a composition formulated for inhalation may be administered using a nasal spray, a nebulizer, a face mask, or a ventilator.

A formulation comprising a composition for nasal or pulmonary deliver may have a pH corresponding to a physiologically acidic nasal pH. The physiologically acidic nasal pH may depend on intact nasal mucosal function. A composition may comprise a pH of about be 6.5±0.5 (5.9 to 7.3) or about 6.7±0.6 (5.3 to 7.6). A composition may comprise a pH of about 3.8-7.7 (mean±SD 5.7±0.9). A composition for nasal or pulmonary deliver may be in the slightly acidic range. The average pH may have an acidity of pH 5.7.

In some embodiments, a formulation for inhalation via nebulization may comprise a pH of about bc 6.5±0.5 (5.9 to 7.3) or about 6.7±0.6 (5.3 to 7.6), or a composition may comprise a pH of about 3.8-7.7 (mean+SD 5.7±0.9).

In some embodiments, a composition may comprise an acid to adjust the pH. For example, a composition may comprise hydrochloric acid, acetic acid, or citric acid. In some embodiments, a composition may comprise a base to adjust the pH. For example, a composition may comprise sodium hydroxide or potassium hydroxide.

In some embodiments, a composition of the present disclosure to treat pulmonary damage (e.g., pulmonary damage associated with radiation therapy, chemotherapy, or both) may be formulated to minimize a chloride ion concentration. A chloride ion concentration may be less than about 1 M, less than about 100 mM, less than about 10 mM, less than about 1 mM, less than about 0.1 mM, or less than about 0.01 mM.

Exemplary mucoadhesive polymer-enzyme inhibitor complexes that are useful within the mucosal formulations and methods of the invention include, but are not limited to: heparin, N-acetyl-cysteine, Carboxymethylcellulose-pepstatin (with anti-pepsin activity); Poly (acrylic acid)-Bowman-Birk inhibitor (anti-chymotrypsin); Poly (acrylic acid)-chymostatin (anti-chymotrypsin); Poly (acrylic acid)-clastatinal (anti-elastase); Carboxymethylcellulose-clastatinal (anti-clastasc); Polycarbophil—clastatinal (anti-clastase); Chitosan—antipain (anti-trypsin); Poly (acrylic acid)—bacitracin (anti-aminopeptidase N); Chitosan—EDTA (anti-aminopeptidase N, anti-carboxypeptidase A); Chitosan—EDTA—antipain (anti-trypsin, anti-chymotrypsin, anti-clastasc).

### Elemental Contaminants

A composition of the present disclosure (e.g., a heparin composition formulated for inhalation) may contain levels of elemental contaminants that are below a threshold limit or may be substantially free of elemental contaminants. In some embodiments, an elemental contaminant may be cadmium (Cd), lead (Pb), arsenic (As), mercury (Hg), cobalt (Co), vanadium (V), nickel (Ni), thallium (TI), gold (Au), palladium (Pd), iridium (Ir), osmium (Os), rhodium (Rh), ruthenium (Ru), selenium (Se), silver (Ag), platinum (Pt), lithium (Li), antimony (Sb), barium (Ba), molybdenum (Mo), copper (Cu), tin (Sn), chromium (Cr), or combinations thereof. The heparin may be purified to remove elemental contaminants by any method known in the art, such as by anion exchange chromatography. In some embodiments, the heparin is purified and optionally dried (e.g., lyophilized) prior to formulating as a pharmaceutical composition. In some embodiments, the pharmaceutical composition is purified.

A heparin composition may comprise a level of Cd that is not more than 30 μg Cd per g heparin (μg/g Cd, or ppm), not more than 15, not more than 10, not more than 7.5, or not more than 6 μg/g Cd. A heparin composition may comprise a level of Cd that is not less than 0.1 and not more than 30 μg Cd per g heparin (μg/g Cd, or ppm), not less than 0.1 and not more than 15, not less than 0.1 and not more than 10, or not less than 0.1 and not more than 7.5 μg Cd per g heparin (μg/g Cd, or ppm). A heparin composition may comprise a level of Cd that is not less than 0 and not more than 30 μg Cd per g heparin (μg/g Cd, or ppm), not more than 15, not more than 10, not more than 7.5, or not more than 6 μg/g Cd. A heparin composition may comprise a level of Cd that is not less than 0.001 and not more than 0.5 μg Cd per g heparin (μg/g Cd, or ppm).

A heparin composition may comprise a level of Pb that is not more than 50 μg Pb per g heparin (μg/g Pb, or ppm), not more than 25 μg/g Pb, not more than 16.7 μg/g Pb, not more than 12.5 μg/g Pb, or not more than 10 μg/g Pb. A heparin composition may comprise a level of Pb that is not less than 0.1 and not more than 50 μg Pb per g heparin (μg/g Pb, or ppm), not less than 0.1 and not more than 25, not less than 0.1 and not more than 16.7, not less than 0.1 and not more than 12.5, or not less than 0.1 and not more than 10 μg Pb per g heparin (μg/g Pb, or ppm). A heparin composition may comprise a level of Pb that is not less than 0 and not more than 50 μg Pb per g heparin (μg/g Pb, or ppm), not more than 25 μg/g Pb, not more than 16.7 μg/g Pb, not more than 12.5 μg/g Pb, or not more than 10 μg/g Pb. A heparin composition may comprise a level of Pb that is not less than 0.001 and not more than 0.5 μg Pb per g heparin (μg/g Pb, or ppm).

A heparin composition may comprise a level of As that is not more than 20 μg As per g heparin (μg/g As, or ppm), not more than 10 μg/g As, not more than 6.7 μg/g As, not more than 5 μg/g As, or not more than 4 μg μg/g As. A heparin composition may comprise a level of As that is not less than 0.1 and not more than 20 μg As per g heparin (μg/g As, or ppm), not less than 0.01 and not more than 5, not less than 0.01 and not more than 4, not less than 0.01 and not more than 2.5 μg As per g heparin (μg/g As, or ppm). A heparin composition may comprise a level of As that is not less than 0 and not more than 20 μg As per g heparin (μg/g As, or ppm), not more than 10 μg/g As, not more than 6.7 μg/g As, not more than 5 μg/g As, or not more than 4 μg μg/g As. A heparin composition may comprise a level of As that is not less than 0.001 and not more than 0.5 μg As per g heparin (μg/g As, or ppm).

A heparin composition may comprise a level of Hg that is not more than 10 μg Hg per g heparin (μg/g Hg, or ppm), not more than 5 μg/g Hg, not more than 3.3 g/g Hg, not more than 2.5 μg/g Hg, or not more than 2 μg/g Hg. A heparin composition may comprise a level of Hg that is not less than 0.1 and not more than 10 μg Hg per g heparin (μg/g Hg, or ppm), not less than 0.01 and not more than 2.5, not less than 0.01 and not more than 1.5, or not less than 0.01 and not more than 0.5 μg Hg per g heparin (μg/g Hg, or ppm). A heparin composition may comprise a level of Hg that is not less than 0 and not more than 10 μg Hg per g heparin (μg/g Hg, or ppm), not more than 5 μg/g Hg, not more than 3.3 μg/g Hg, not more than 2.5 μg/g Hg, or not more than 2 μg/g Hg. A heparin composition may comprise a level of Hg that is not less than 0.001 and not more than 0.5 μg Hg per g heparin (μg/g Hg, or ppm).

A heparin composition may comprise a level of Co that is not more than 30 μg Co per g heparin (μg/g Co, or ppm), not more than 15 μg/g Co, not more than 10 μg/g Co, not more than 7.5 μg/g Co, or not more than 6 μg/g Co. A heparin composition may comprise a level of Co that is not less than 0.1 and not more than 30 μg Co per g heparin (μg/g Co, or ppm), not less than 0.01 and not more than 7.5, not less than 0.01 and not more than 5, or not less than 0.01 and not more than 2.5 μg Co per g heparin (μg/g Co, or ppm). A heparin composition may comprise a level of Co that is not less than 0 and not more than 30 μg Co per g heparin (μg/g Co, or ppm), not more than 15 μg/g Co, not more than 10 μg/g Co, not more than 7.5 μg/g Co, or not more than 6 μg/g Co. A heparin composition may comprise a level of Co that is not less than 0.001 and not more than 0.5 μg Co per g heparin (μg/g Co, or ppm).

A heparin composition may comprise a level of V that is not more than 10 μg V per g heparin (μg/g V, or ppm), not more than 5 μg/g V, not more than 3.3 μg/g V, not more than 2.5 μg/g V, or not more than 2 μg/g V. A heparin composition may comprise a level of V that is not less than 0.1 and not more than 10 μg V per g heparin (μg/g V, or ppm), not less than 0.01 and not more than 2.5, not less than 0.01 and not more than 1.5, or not less than 0.01 and not more than 0.5 μg V per g heparin (μg/g V, or ppm). A heparin composition may comprise a level of V that is not less than 0 and not more than 10 μg V per g heparin (μg/g V, or ppm), not more than 5 μg/g V, not more than 3.3 μg/g V, not more than 2.5 μg/g V, or not more than 2 μg/g V. A heparin composition may comprise a level of V that is not less than 0.001 and not more than 0.5 μg V per g heparin (μg/g V, or ppm).

A heparin composition may comprise a level of Ni that is not more than 50 μg Ni per g heparin (μg/g Ni, or ppm), not more than 25 μg/g Ni, not more than 16.7 μg/g Ni, not more than 12.5 μg/g Ni, or not more than 10 μg/g Ni. A heparin composition may comprise a level of Ni that is not less than 0.1 and not more than 50 μg Ni per g heparin (μg/g Ni, or ppm), not less than 0.1 and not more than 12.5, not less than 0.01 and not more than 7.5, or not less than 0.01 and not more than 5 μg Ni per g heparin (μg/g Ni, or ppm). A heparin composition may comprise a level of Ni that is not less than 0 and not more than 50 μg Ni per g heparin (μg/g Ni, or ppm), not more than 25 μg/g Ni, not more than 16.7 μg/g Ni, not more than 12.5 μg/g Ni, or not more than 10 μg/g Ni. A heparin composition may comprise a level of Ni that is not less than 0.001 and not more than 0.5 μg Ni per g heparin (μg/g Ni, or ppm).

A heparin composition may comprise a level of T1 that is not more than 80 μg T1 per g heparin (μg/g T1, or ppm), not more than 40 μg/g T1, not more than 26.7 μg/g T1, not more than 20 μg/g T1, or not more than 16 μg μg/g T1. A heparin composition may comprise a level of TI that is not less than 0.1 and not more than 80 μg T1 per g heparin (μg/g T1, or ppm), not less than 0.1 and not more than 40, not less than 0.1 and not more than 26.7, not less than 0.1 and not more than 20, or not less than 0.1 and not more than 16 μg TI per g heparin (μg/g T1, or ppm). A heparin composition may comprise a level of T1 that is not less than 0 and not more than 80 μg T1 per g heparin (μg/g T1, or ppm), not more than 40 μg/g T1, not more than 26.7 μg/g T1, not more than 20 μg/g T1, or not more than 16 μg μg/g T1. A heparin composition may comprise a level of T1 that is not less than 0.001 and not more than 0.5 μg TI per g heparin (μg/g T1, or ppm).

A heparin composition may comprise a level of Au that is not more than 10 μg Au per g heparin (μg/g Au, or ppm), not more than 5 μg/g Au, not more than 3.3 μg/g Au, not more than 2.5 μg/g Au, or not more than 2 μg/g Au. A heparin composition may comprise a level of Au that is not less than 0.1 and not more than 10 μg Au per g heparin (μg/g Au, or ppm), not less than 0.01 and not more than 7.5, not less than 0.01 and not more than 5, not less than 0.01 and not more than 2.5, or not less than 0.01 and not more than 1 μg Au per g heparin (μg/g Au, or ppm). A heparin composition may comprise a level of Au that is not less than 0 and more than 10 μg Au per g heparin (μg/g Au, or ppm), not more than 5 μg/g Au, not more than 3.3 μg/g Au, not more than 2.5 μg/g Au, or not more than 2 μg/g Au. A heparin composition may comprise a level of Au that is not less than 0.001 and not more than 0.5 μg Au per g heparin (μg/g Au, or ppm).

A heparin composition may comprise a level of Pd that is not more than 10 μg Pd per g heparin (μg/g Pd, or ppm), not more than 5 μg/g Pd, not more than 3.3 μg/g Pd, not more than 2.5 μg/g Pd, or not more than 2 μg/g Pd. A heparin composition may comprise a level of Pd that is not less than 1 and not more than 10 μg Pd per g heparin (μg/g Pd, or ppm), not less than 0.01 and not more than 2.5, not less than 0.01 and not more than 1.5, or not less than 0.01 and not more than 0.5 μg Pd per g heparin (μg/g Pd, or ppm). A heparin composition may comprise a level of Pd that is not less than 0 and not more than 10 μg Pd per g heparin (μg/g Pd, or ppm), not more than 5 μg/g Pd, not more than 3.3 μg/g Pd, not more than 2.5 μg/g Pd, or not more than 2 μg/g Pd. A heparin composition may comprise a level of Pd that is not less than 0.001 and not more than 0.5 μg Pd per g heparin (μg/g Pd, or ppm).

A heparin composition may comprise a level of Ir that is not more than 10 μg Ir per g heparin (μg/g Ir, or ppm), not more than 5 μg/g Ir, not more than 3.3 μg/g Ir, not more than 2.5 μg/g Ir, or not more than μg/g Ir. A heparin composition may comprise a level of Ir that is not less than 1 and not more than 10 μg Ir per g heparin (μg/g Ir, or ppm), not less than 0.01 and not more than 2.5, not less than 0.01 and not more than 1.5, or not less than 0.01 and not more than 0.5 μg Ir per g heparin (μg/g Ir, or ppm). A heparin composition may comprise a level of Ir that is not less than 0 and not more than 10 μg Ir per g heparin (μg/g Ir, or ppm), not more than 5 μg/g Ir, not more than 3.3 μg/g Ir, not more than 2.5 μg/g Ir, or not more than μg/g Ir. A heparin composition may comprise a level of Ir that is not less than 0.001 and not more than 0.5 μg Ir per g heparin (μg/g Ir, or ppm).

A heparin composition may comprise a level of Os that is not more than 10 μg Os per g heparin (μg/g Os, or ppm), not more than 5 μg/g Os, not more than 3.3 μg/g Os, not more than 2.5 μg/g Os, or not more than 2 μg/g Os. A heparin composition may comprise a level of Os that is not less than 1 and not more than 10 μg Os per g heparin (μg/g Os, or ppm), not less than 0.01 and not more than 2.5, not less than 0.01 and not more than 1.5, or not less than 0.01 and not more than 0.5 μg Os per g heparin (μg/g Os, or ppm). A heparin composition may comprise a level of Os that is not less than 0 and not more than 10 μg Os per g heparin (μg/g Os, or ppm), not more than 5 μg/g Os, not more than 3.3 μg/g Os, not more than 2.5 μg/g Os, or not more than 2 μg/g Os. A heparin composition may comprise a level of Os that is not less than 0.001 and not more than 0.5 μg Os per g heparin (μg/g Os, or ppm).

A heparin composition may comprise a level of Rh that is not more than 10 μg Rh per g heparin (μg/g Rh, or ppm), not more than 5 μg/g Rh, not more than 3.3 μg/g Rh, not more than 2.5 μg/g Rh, or not more than 2 μg/g Rh. A heparin composition may comprise a level of Rh that is not less than 1 and not more than 10 μg Rh per g heparin (μg/g Rh, or ppm), not less than 0.01 and not more than 2.5, not less than 0.01 and not more than 1.5, or not less than 0.01 and not more than 0.5 μg Rh per g heparin (μg/g Rh, or ppm). A heparin composition may comprise a level of Rh that is not less than 0 and not more than 10 μg Rh per g heparin (μg/g Rh, or ppm), not more than 5 μg/g Rh, not more than 3.3 μg/g Rh, not more than 2.5 μg/g Rh, or not more than 2 μg/g Rh. A heparin composition may comprise a level of Rh that is not less than 0.001 and not more than 0.5 μg Rh per g heparin (μg/g Rh, or ppm).

A heparin composition may comprise a level of Ru that is not more than 10 μg Ru per g heparin (μg/g Ru, or ppm), not more than 5 μg/g Ru, not more than 3.3 μg/g Ru, not more than 2.5 μg/g Ru, or not more than 2 μg/g Ru. A heparin composition may comprise a level of Ru that is not less than 1 and not more than 10 μg Ru per g heparin (μg/g Ru, or ppm), not less than 0.01 and not more than 2.5, not less than 0.01 and not more than 1.5, or not less than 0.01 and not more than 0.5 μg Ru per g heparin (μg/g Ru, or ppm). A heparin composition may comprise a level of Ru that is not more than 10 μg Ru per g heparin (μg/g Ru, or ppm), not more than 5 μg/g Ru, not more than 3.3 μg/g Ru, not more than 2.5 μg/g Ru, or not more than 2 μg/g Ru. A heparin composition may comprise a level of Ru that is not less than 0.001 and not more than 0.5 μg Ru per g heparin (μg/g Ru, or ppm).

A heparin composition may comprise a level of Se that is not more than 1300 μg Se per g heparin (μg/g Se, or ppm), not more than 650 μg/g Se, not more than 433.3 μg/g Se, not more than 325 μg/g Se, or not more than 260 μg/g Se. A heparin composition may comprise a level of Se that is not less than 10 and not more than 1300 μg Se per g heparin (μg/g Se, or ppm), not less than 10 and not more than 650, not less than 10 and not more than 433.3, not less than 10 and not more than 325, or not less than 10 and not more than 260 μg Se per g heparin (μg/g Sc, or ppm). A heparin composition may comprise a level of Se that is not less than 0 and not more than 1300 μg Se per g heparin (μg/g Se, or ppm), not more than 650 μg/g Se, not more than 433.3 μg/g Se, not more than 325 μg/g Se, or not more than 260 μg/g Se. A heparin composition may comprise a level of Se that is not less than 0.001 and not more than 0.5 μg Se per g heparin (μg/g Sc, or ppm).

A heparin composition may comprise a level of Ag that is not more than 70 μg Ag per g heparin (μg/g Ag, or ppm), not more than 35 μg/g Ag, not more than 23.3 μg/g Ag, not more than 17.5 μg/g Ag, or not more than 14 μg/g Ag. A heparin composition may comprise a level of Ag that is not less than 1 and not more than 70 μg Ag per g heparin (μg/g Ag, or ppm), not less than 0.1 and not more than 25, not less than 0.1 and not more than 17.5, not less than 0.1 and not more than 10, or not less than 0.01 and not more than 5 μg Ag per g heparin (μg/g Ag, or ppm). A heparin composition may comprise a level of Ag that is not less than 0 and not more than 70 μg Ag per g heparin (μg/g Ag, or ppm), not more than 35 μg/g Ag, not more than 23.3 μg/g Ag, not more than 17.5 μg/g Ag, or not more than 14 μg/g Ag. A heparin composition may comprise a level of Ag that is not less than 0.001 and not more than 0.5 μg Ag per g heparin (μg/g Ag, or ppm).

A heparin composition may comprise a level of Pt that is not more than 10 μg Pt per g heparin (μg/g Pt, or ppm), not more than 5 μg/g Pt, not more than 3.3 μg/g Pt, not more than 2.5 ug/g Pt, or not more than 2 μg μg/g Pt. A heparin composition may comprise a level of Pt that is not less than 1 and not more than 10 μg Pt per g heparin (μg/g Pt, or ppm), not less than 0.01 and not more than 2.5, not less than 0.01 and not more than 1.5, or not less than 0.01 and not more than 0.5 μg Pt per g heparin (μg/g Pt, or ppm). A heparin composition may comprise a level of Pt that is not less than 0 and not more than 10 μg Pt per g heparin (μg/g Pt, or ppm), not more than 5 μg/g Pt, not more than 3.3 μg/g Pt, not more than 2.5 μg/g Pt, or not more than 2 μg ug/g Pt. A heparin composition may comprise a level of Pt that is not less than 0.001 and not more than 0.5 μg Pt per g heparin (μg/g Pt, or ppm).

A heparin composition may comprise a level of Li that is not more than 250 μg Li per g heparin (μg/g Li, or ppm), not more than 125 μg/g Li, not more than 83.3 μg/g Li, not more than 62.5 μg/g Li, or not more than 50 μg/g Li. A heparin composition may comprise a level of Li that is not less than 1 and not more than 250 μg Li per g heparin (μg/g Li, or ppm), not less than 1 and not more than 100, not less than 0.1 and not more than 17.5, not less than 0.1 and not more than 10, or not less than 0.01 and not more than 5 μg Li per g heparin (μg/g Li, or ppm). A heparin composition may comprise a level of Li that is not less than 0 and not more than 250 μg Li per g heparin (μg/g Li, or ppm), not more than 125 μg/g Li, not more than 83.3 μg/g Li, not more than 62.5 μg/g Li, or not more than 50 μg/g Li. A heparin composition may comprise a level of Li that is not less than 0.001 and not more than 0.5 μg Li per g heparin (μg/g Li, or ppm).

A heparin composition may comprise a level of Sb that is not more than 200 μg Sb per g heparin (μg/g Sb, or ppm), not more than 100 μg/g Sb, not more than 66.7 μg/g Sb, not more than 50 μg/g Sb, or not more than 40 μg/g Sb. A heparin composition may comprise a level of Sb that is not less than 1 and more than 200 μg Sb per g heparin (μg/g Sb, or ppm), not less than 1 and not more than 50, not less than 0.1 and not more than 25, or not less than 0.1 and not more than 10 μg Sb per g heparin (μg/g Sb, or ppm). A heparin composition may comprise a level of Sb that is not less than 0 and not more than 200 μg Sb per g heparin (μg/g Sb, or ppm), not more than 100 μg/g Sb, not more than 66.7 μg/g Sb, not more than 50 μg/g Sb, or not more than 40 μg/g Sb. A heparin composition may comprise a level of Sb that is not less than 0.001 and not more than 0.5 μg Sb per g heparin (μg/g Sb, or ppm).

A heparin composition may comprise a level of Ba that is not more than 3000 μg Ba per g heparin (μg/g Ba, or ppm), not more than 1500 μg/g Ba, not more than 1000 μg/g Ba, not more than 750 μg/g Ba, or not more than 600 μg/g Ba. A heparin composition may comprise a level of Ba that is not less than 10 and not more than 3000 μg Ba per g heparin (μg/g Ba, or ppm), not less than 10 and not more than 1000, not less than 10 and not more than 750, not less than 10 and not more than 500, or not less than 10 and not more than 250 μg Ba per g heparin (μg/g Ba, or ppm). A heparin composition may comprise a level of Ba that is not less than 0 and not more than 3000 μg Ba per g heparin (μg/g Ba, or ppm), not more than 1500 μg/g Ba, not more than 1000 μg/g Ba, not more than 750 μg/g Ba, or not more than 600 μg/g Ba. A heparin composition may comprise a level of Ba that is not less than 0.001 and not more than 0.5 μg Ba per g heparin (μg/g Ba, or ppm).

A heparin composition may comprise a level of Mo that is not more than 100 μg Mo per g heparin (μg/g Mo, or ppm), not more than 50 μg/g Mo, not more than 33.3 μg/g Mo, not more than 25 μg/g Mo, or not more than 20 μg/g Mo. A heparin composition may comprise a level of Mo that is not less than 1 and not more than 100 μg Mo per g heparin (μg/g Mo, or ppm), not less than 1 and not more than 250, not less than 1 and not more than 150, or not less than 0.1 and not more than 25 μg Mo per g heparin (μg/g Mo, or ppm). A heparin composition may comprise a level of Mo that is not less than 0 and not more than 100 μg Mo per g heparin (μg/g Mo, or ppm), not more than 50 μg/g Mo, not more than 33.3 μg/g Mo, not more than 25 μg/g Mo, or not more than 20 μg/g Mo. A heparin composition may comprise a level of Mo that is not less than 0.001 and not more than 0.5 μg Mo per g heparin (μg/g Mo, or ppm).

A heparin composition may comprise a level of Cu that is not more than 300 μg Cu per g heparin (μg/g Cu, or ppm), not more than 150 μg/g Cu, not more than 100 μg/g Cu, not more than 75 μg/g Cu, or not more than 60 μg/g Cu. A heparin composition may comprise a level of Cu that is not less than 1 and not more than 300 μg Cu per g heparin (μg/g Cu, or ppm), not less than 1 and not more than 75, not less than 0.1 and not more than 50, or not less than 0.1 and not more than 25 μg Cu per g heparin (μg/g Cu, or ppm). A heparin composition may comprise a level of Cu that is not less than 0 and not more than 300 μg Cu per g heparin (μg/g Cu, or ppm), not more than 150 μg/g Cu, not more than 100 μg/g Cu, not more than 75 μg/g Cu, or not more than 60 μg/g Cu. A heparin composition may comprise a level of Cu that is not less than 0.001 and not more than 0.5 μg Cu per g heparin (μg/g Cu, or ppm).

A heparin composition may comprise a level of Sn that is not more than 600 μg Sn per g heparin (μg/g Sn, or ppm), not more than 300 μg/g Sn, not more than 200 μg/g Sn, not more than 150 μg/g Sn, or not more than 120 μg/g Sn. A heparin composition may comprise a level of Sn that is not less than 1 and not more than 600 μg Sn per g heparin (μg/g Sn, or ppm), not less than 1 and not more than 150, not less than 0.1 and not more than 75, or not less than 0.1 and not more than 50 μg Sn per g heparin (μg/g Sn, or ppm). A heparin composition may comprise a level of Sn that is not less than 0 and not more than 600 μg Sn per g heparin (μg/g Sn, or ppm), not more than 300 μg/g Sn, not more than 200 μg/g Sn, not more than 150 μg/g Sn, or not more than 120 μg/g Sn. A heparin composition may comprise a level of Sn that is not less than 0.001 and not more than 0.5 μg Sn per g heparin (μg/g Sn, or ppm).

A heparin composition may comprise a level of Cr that is not more than 30 μg Cr per g heparin (μg/g Cr, or ppm), not more than 15 μg/g Cr, not more than 10 μg/g Cr, not more than 7.5 μg/g Cr, or not more than 6 μg/g Cr. A heparin composition may comprise a level of Cr that is not less than 0.1 and not more than 30 μg Cr per g heparin (μg/g Cr, or ppm), not less than 0.1 and not more than 75, not less than 0.1 and not more than 25, not less than 0.1 and not more than 10, not less than 0.01 and not more than 7.5 μg Cr per g heparin (μg/g Cr, or ppm). A heparin composition may comprise a level of Cr that is not less than 0 and not more than 30 μg Cr per g heparin (μg/g Cr, or ppm), not more than 15 μg/g Cr, not more than 10 μg/g Cr, not more than 7.5 μg/g Cr, or not more than 6 μg/g Cr. A heparin composition may comprise a level of Cr that is not less than 0.001 and not more than 0.5 μg Cr per g heparin (μg/g Cr, or ppm).

### Endotoxin-Free Formulations

In some embodiments, an endotoxin-free formulation may be a formulation which contains a Y2-receptor-binding peptide and one or more mucosal delivery enhancing agents. A composition of the present disclosure may be substantially free of endotoxins and/or related pyrogenic substances. Endotoxins include toxins that are confined inside a microorganism and are released only when the microorganisms are broken down or die. Pyrogenic substances include fever-inducing, thermostable substances (glycoproteins) from the outer membrane of bacteria and other microorganisms. Both of these substances can cause fever, hypotension and shock if administered to humans. Producing formulations that are endotoxin-free can require special equipment, expert artisans, and can be significantly more expensive than making formulations that are not endotoxin-free.

### Mucolytic and Mucus-Clearing Agents and Methods

Effective delivery of therapeutic agents via nasal or pulmonary administration may take into account the decreased drug transport rate across the protective mucus lining of the nasal mucosa, in addition to drug loss due to binding to glycoproteins of the mucus layer. Normal mucus is a viscoelastic, gel-like substance consisting of water, electrolytes, mucins, macromolecules, and sloughed epithelial cells. It serves primarily as a cytoprotective and lubricative covering for the underlying mucosal tissues. Mucus is secreted by randomly distributed secretory cells located in the nasal epithelium and in other mucosal epithelia. The structural unit of mucus is mucin. This glycoprotein is mainly responsible for the viscoelastic nature of mucus, although other macromolecules may also contribute to this property. In airway mucus, such macromolecules include locally produced secretory IgA, IgM, IgE, lysozyme, and bronchotransferrin, which also play an important role in host defense mechanisms.

The coordinate administration methods of the instant invention optionally incorporate effective mucolytic or mucus-clearing agents, which serve to degrade, thin or clear mucus from intranasal mucosal surfaces to facilitate absorption and/or adsorption of intranasally administered biotherapeutic agents. Within these methods, a mucolytic or mucus-clearing agent is coordinately administered as an adjunct compound to enhance intranasal delivery of the biologically active agent. Alternatively, an effective amount of a mucolytic or mucus-clearing agent is incorporated as a processing agent within a multi-processing method of the invention, or as an additive within a combinatorial formulation of the invention, to provide an improved formulation that enhances intranasal delivery of biotherapeutic compounds by reducing the barrier effects of intranasal mucus.

A variety of mucolytic or mucus-clearing agents are available for incorporation within the methods and compositions of the invention. Based on their mechanisms of action, mucolytic and mucus clearing agents can often be classified into the following groups: proteases (e.g., pronase, papain) that cleave the protein core of mucin glycoproteins; sulfhydryl compounds that split mucoprotein disulfide linkages; and detergents (e.g., Triton X-100, Tween 20) that break non-covalent bonds within the mucus. Additional compounds in this context include, but are not limited to, bile salts and surfactants, for example, sodium deoxycholate, sodium taurodeoxycholate, sodium glycocholate, and lysophosphatidylcholine.

The effectiveness of bile salts in causing structural breakdown of mucus is in the order deoxycholate>taurocholate>glycocholate. Other effective agents that reduce mucus viscosity or adhesion to enhance intranasal delivery according to the methods of the invention include, e.g., short-chain fatty acids, and mucolytic agents that work by chelation, such as N-acylcollagen peptides, bile acids, and saponins (the latter function in part by chelating Ca²⁺ and/or Mg²⁺ which play an important role in maintaining mucus layer structure).

Additional mucolytic agents for use within the methods and compositions of the invention include N-acetyl-L-cysteine (ACS), a potent mucolytic agent that reduces both the viscosity and adherence of bronchopulmonary mucus and is reported to modestly increase nasal bioavailability of human growth hormone in anesthetized rats (from 7.5 to 12.2%). These and other mucolytic or mucus-clearing agents are contacted with the nasal mucosa, typically in a concentration range of about 0.2 to 20 mM, coordinately with administration of the biologically active agent, to reduce the polar viscosity and/or elasticity of intranasal mucus.

Still other mucolytic or mucus-clearing agents may be selected from a range of glycosidase enzymes, which are able to cleave glycosidic bonds within the mucus glycoprotein. α-amylase and β-amylase are representative of this class of enzymes, although their mucolytic effect may be limited. In contrast, bacterial glycosidases which allow these microorganisms to permeate mucus layers of their hosts.

For combinatorial use with most biologically active agents within the invention, including peptide and protein therapeutics, non-ionogenic detergents are generally also useful as mucolytic or mucus-clearing agents. These agents typically will not modify or substantially impair the activity of therapeutic polypeptides.

### Ciliostatic Agents and Methods

Because the self-cleaning capacity of certain mucosal tissues (e.g., pulmonary mucosal tissues) by mucociliary clearance is necessary as a protective function (e.g., to remove dust, allergens, and bacteria), it has been generally considered that this function should not be substantially impaired by mucosal medications. Mucociliary transport in the respiratory tract is a particularly important defense mechanism against infections. To achieve this function, ciliary beating in the nasal and airway passages moves a layer of mucus along the mucosa to removing inhaled particles and microorganisms.

Ciliostatic agents find use within the methods and compositions of the invention to increase the residence time of mucosally (e.g., intranasally or pulmonary) administered biologically active agents disclosed herein. In particular, the delivery these agents within the methods and compositions of the invention is significantly enhanced in certain aspects by the coordinate administration or combinatorial formulation of one or more ciliostatic agents that function to reversibly inhibit ciliary activity of mucosal cells, to provide for a temporary, reversible increase in the residence time of the mucosally administered active agent(s). For use within these aspects of the invention, the foregoing ciliostatic factors, either specific or indirect in their activity, are all candidates for successful employment as ciliostatic agents in appropriate amounts (depending on concentration, duration and mode of delivery) such that they yield a transient (i.e., reversible) reduction or cessation of mucociliary clearance at a mucosal site of administration to enhance delivery of biologically active agents disclosed herein, without unacceptable adverse side effects.

Within more detailed aspects, a specific ciliostatic factor is employed in a combined formulation or coordinate administration protocol with one or more Y2 receptor-binding peptide proteins, analogs and mimetics, and/or other biologically active agents disclosed herein. Various bacterial ciliostatic factors isolated and characterized in the literature may be employed within these embodiments of the invention. Ciliostatic factors from the bacterium Pseudomonas aeruginosa include a phenazine derivative, a pyo compound (2-alkyl-4-hydroxyquinolines), and a rhamnolipid (also known as a hemolysin). The pyo compound produced ciliostasis at concentrations of 50 μg/ml and without obvious ultrastructural lesions. The phenazine derivative also inhibited ciliary motility but caused some membrane disruption, although at substantially greater concentrations of 400 μg/ml. Limited exposure of tracheal explants to the rhamnolipid resulted in ciliostasis, which was associated with altered ciliary membranes. More extensive exposure to rhamnolipid was associated with removal of dynein arms from axonemes.

### Surface Active Agents and Methods

Within more detailed aspects of the invention, one or more membrane penetration-enhancing agents may be employed within a mucosal delivery method or formulation of the invention to enhance mucosal delivery biologically active agents disclosed herein. Membrane penetration enhancing agents in this context can be selected from: (i) a surfactant, (ii) a bile salt, (iii) a phospholipid additive, mixed micelle, liposome, or carrier, (iv) an alcohol, (v) an enamine, (vi) an NO donor compound, (vii) a long-chain amphipathic molecule (viii) a small hydrophobic penetration enhancer; (ix) sodium or a salicylic acid derivative; (x) a glycerol ester of acetoacetic acid (xi) a cyclodextrin or beta-cyclodextrin derivative, (xii) a medium-chain fatty acid, (xiii) a chelating agent, (xiv) an amino acid or salt thereof, (xv) an N-acetylamino acid or salt thereof, (xvi) an enzyme degradative to a selected membrane component, (xvii) an inhibitor of fatty acid synthesis, or (xviii) an inhibitor of cholesterol synthesis; or (xix) any combination of the membrane penetration enhancing agents recited in (i) (xix).

Certain surface-active agents are readily incorporated within the mucosal delivery formulations and methods of the invention as mucosal absorption and/or adsorption enhancing agents. These agents, which may be coordinately administered or combinatorially formulated with other biologically active agents disclosed herein, may be selected from a broad assemblage of known surfactants. Surfactants, which generally fall into three classes: (1) nonionic polyoxyethylene ethers; (2) bile salts such as sodium glycocholate (SGC) and deoxycholate (DOC); and (3) derivatives of fusidic acid such as sodium taurodihydrofusidate (STDHF). The mechanisms of action of these various classes of surface-active agents typically include solubilization of the biologically active agent. For proteins and peptides which often form aggregates, the surface active properties of these absorption and/or adsorption promoters can allow interactions with proteins such that smaller units such as surfactant coated monomers may be more readily maintained in solution. Examples of other surface-active agents are L-α-Phosphatidylcholine Didecanoyl (DDPC) polysorbate 80 and polysorbate 20. These monomers are presumably more transportable units than aggregates. A second potential mechanism is the protection of the peptide or protein from proteolytic degradation by proteases in the mucosal environment. Both bile salts and some fusidic acid derivatives reportedly inhibit proteolytic degradation of proteins by nasal homogenates at concentrations less than or equivalent to those required to enhance protein absorption and/or adsorption. This protease inhibition may be especially important for peptides with short biological half-lives.

### Vasodilator Agents and Methods

While generally it is intended the formulations of this invention remain on the pulmonary mucosa to perform their damage treatment action, yet another class of absorption and/or adsorption-promoting agents that shows beneficial utility within the coordinate administration and combinatorial formulation methods and compositions of the invention are vasoactive compounds, more specifically vasodilators. These compounds function within the invention to modulate the structure and physiology of the submucosal vasculature, increasing the transport rate of biologically active agents into or through the mucosal epithelium and/or to specific target tissues or compartments (e.g., the systemic circulation or central nervous system.).

Vasodilator agents for use within the invention typically cause submucosal blood vessel relaxation by either a decrease in cytoplasmic calcium, an increase in nitric oxide (NO) or by inhibiting myosin light chain kinase. They are generally divided into 9 classes: calcium antagonists, potassium channel openers, ACE inhibitors, angiotensin-II receptor antagonists, α-adrenergic and imidazole receptor antagonists, β1-adrenergic agonists, phosphodiesterase inhibitors, cicosanoids and NO donors.

Despite chemical differences, the pharmacokinetic properties of calcium antagonists are similar. Absorption and/or adsorption into the systemic circulation is high, and these agents therefore undergo considerable first-pass metabolism by the liver, resulting in individual variation in pharmacokinetics. Except for the newer drugs of the dihydropyridine type (amlodipine, felodipine, isradipine, nilvadipine, nisoldipine and nitrendipine), the half-life of calcium antagonists is short. Therefore, to maintain an effective drug concentration for many of these may require delivery by multiple dosing, or controlled release formulations, as described elsewhere herein. Treatment with the potassium channel opener minoxidil may also be limited in manner and level of administration due to potential adverse side effects.

ACE inhibitors prevent conversion of angiotensin-I to angiotensin-II, and are most effective when renin production is increased. Since ACE is identical to kininase-II, which inactivates the potent endogenous vasodilator bradykinin, ACE inhibition causes a reduction in bradykinin degradation. ACE inhibitors provide the added advantage of cardioprotective and cardioreparative effects, by preventing and reversing cardiac fibrosis and ventricular hypertrophy in animal models. The predominant elimination pathway of most ACE inhibitors is via renal excretion. Therefore, renal impairment is associated with reduced elimination and a dosage reduction of 25 to 50% is recommended in patients with moderate to severe renal impairment.

With regard to NO donors, these compounds are particularly useful within the invention for their additional effects on mucosal permeability. In addition to the above-noted NO donors, complexes of NO with nucleophiles called NO/nucleophiles, or NONOates, spontaneously and nonenzymatically release NO when dissolved in aqueous solution at physiologic pH. In contrast, nitro vasodilators such as nitroglycerin require specific enzyme activity for NO release. NONOates release NO with a defined stoichiometry and at predictable rates ranging from <3 minutes for diethylamine/NO to approximately 20 hours for diethylenetriamine/NO (DETANO).

### Polymeric Delivery Vehicles and Methods

Within certain aspects of the invention, biologically active agents disclosed herein, and delivery-enhancing agents as described above, are, individually or combinatorially, incorporated within a mucosally (e.g., nasally or pulmonary) administered formulation that includes a biocompatible polymer functioning as a carrier or base. Such polymer carriers include polymeric powders, matrices or microparticulate delivery vehicles, among other polymer forms. The polymer can be of plant, animal, or synthetic origin. Often the polymer is crosslinked. In other embodiments, the polymer is chemically modified with an inhibitor of enzymes or other agents which may degrade or inactivate the biologically active agent(s) and/or delivery enhancing agent(s). In certain formulations, the polymer is a partially or completely water insoluble but water swellable polymer, e.g., a hydrogel. Polymers useful in this aspect of the invention are desirably water interactive and/or hydrophilic in nature to absorb significant quantities of water, and they often form hydrogels when placed in contact with water or aqueous media for a period of time sufficient to reach equilibrium with water. In more detailed embodiments, the polymer is a hydrogel which, when placed in contact with excess water, absorbs at least two times its weight of water at equilibrium when exposed to water at room temperature.

Drug delivery systems based on biodegradable polymers are preferred in many biomedical applications because such systems are broken down either by hydrolysis or by enzymatic reaction into non-toxic molecules. The rate of degradation is controlled by manipulating the composition of the biodegradable polymer matrix. These types of systems can therefore be employed in certain settings for long-term release of biologically active agents. Biodegradable polymers such as poly (glycolic acid) (PGA), poly-(lactic acid) (PLA), and poly (D,L-lactic-co-glycolic acid) (PLGA), have received considerable attention as possible drug delivery carriers, since the degradation products of these polymers have been found to have low toxicity. During the normal metabolic function of the body these polymers degrade into carbon dioxide and water. These polymers have also exhibited excellent biocompatibility.

For prolonging the biological activity of biologically active agents disclosed herein, as well as optional delivery-enhancing agents, these agents may be incorporated into polymeric matrices, e.g., polyorthoesters, polyanhydrides, or polyesters. This yields sustained activity and release of the active agent(s), e.g., as determined by the degradation of the polymer matrix. Although the encapsulation of biotherapeutic molecules inside synthetic polymers may stabilize them during storage and delivery, the largest obstacle of polymer-based release technology is the activity loss of the therapeutic molecules during the formulation processes that often involve heat, sonication or organic solvents.

Absorption and/or adsorption-promoting polymers contemplated for use within the invention may include derivatives and chemically or physically modified versions of the foregoing types of polymers, in addition to other naturally occurring or synthetic polymers, gums, resins, and other agents, as well as blends of these materials with each other or other polymers, so long as the alterations, modifications or blending do not adversely affect the desired properties, such as water absorption and/or adsorption, hydrogel formation, and/or chemical stability for useful application. In more detailed aspects of the invention, polymers such as nylon, acrylan and other normally hydrophobic synthetic polymers may be sufficiently modified by reaction to become water swellable and/or form stable gels in aqueous media.

Absorption and/or adsorption-promoting polymers of the invention may include polymers from the group of homo- and copolymers based on various combinations of the following vinyl monomers: acrylic and methacrylic acids, acrylamide, methacrylamide, hydroxyethylacrylate or methacrylate, vinylpyrrolidones, as well as polyvinylalcohol and its co- and terpolymers, polyvinylacetate, its co- and terpolymers with the above listed monomers and 2-acrylamido-2-methyl-propanesulfonic acid (AMPS.RTM.). Very useful are copolymers of the above listed monomers with copolymerizable functional monomers such as acryl or methacryl amide acrylate or methacrylate esters where the ester groups are derived from straight or branched chain alkyl, aryl having up to four aromatic rings which may contain alkyl substituents of 1 to 6 carbons; steroidal, sulfates, phosphates or cationic monomers such as N,N-dimethylaminoalkyl(meth)acrylamide, dimethylaminoalkyl(meth)acrylate, (meth)acryloxyalkyltrimethylammonium chloride, (meth)acryloxyalkyldimethylbenzyl ammonium chloride.

Additional absorption and/or adsorption-promoting polymers for use within the invention are those classified as dextrans, dextrins, and from the class of materials classified as natural gums and resins, or from the class of natural polymers such as processed collagen, chitin, chitosan, pullalan, zooglan, alginates and modified alginates such as “Kelcoloid” (a polypropylene glycol modified alginate) gellan gums such as “Kelocogel”, Xanathan gums such as “Keltrol”, estastin, alpha hydroxy butyrate and its copolymers, hyaluronic acid and its derivatives, polylactic and glycolic acids.

A very useful class of polymers applicable within the instant invention are olefinically-unsaturated carboxylic acids containing at least one activated carbon-to-carbon olefinic double bond, and at least one carboxyl group; that is, an acid or functional group readily converted to an acid containing an olefinic double bond which readily functions in polymerization because of its presence in the monomer molecule, either in the alpha-beta position with respect to a carboxyl group, or as part of a terminal methylene grouping. Olefinically-unsaturated acids of this class include such materials as the acrylic acids typified by the acrylic acid itself, alpha-cyano acrylic acid, beta methylacrylic acid (crotonic acid), alpha-phenyl acrylic acid, beta-acryloxy propionic acid, cinnamic acid, p-chloro cinnamic acid, 1-carboxy-4-phenyl butadiene-1,3, itaconic acid, citraconic acid, mesaconic acid, glutaconic acid, aconitic acid, maleic acid, fumaric acid, and tricarboxy ethylene. As used herein, the term “carboxylic acid” includes the polycarboxylic acids and those acid anhydrides, such as maleic anhydride, wherein the anhydride group is formed by the elimination of one molecule of water from two carboxyl groups located on the same carboxylic acid molecule.

Representative acrylates useful as absorption and/or adsorption-promoting agents within the invention include methyl acrylate, ethyl acrylate, propyl acrylate, isopropyl acrylate, butyl acrylate, isobutyl acrylate, methyl methacrylate, methyl ethacrylate, ethyl methacrylate, octyl acrylate, heptyl acrylate, octyl methacrylate, isopropyl methacrylate, 2-ethylhexyl methacrylate, nonyl acrylate, hexyl acrylate, n-hexyl methacrylate, and the like. Higher alkyl acrylic esters are decyl acrylate, isodecyl methacrylate, lauryl acrylate, stearyl acrylate, behenyl acrylate and melissyl acrylate and methacrylate versions thereof. Mixtures of two or three or more long chain acrylic esters may be successfully polymerized with one of the carboxylic monomers. Other comonomers include olefins, including alpha olefins, vinyl ethers, vinyl esters, and mixtures thereof.

Other vinylidene monomers, including the acrylic nitriles, may also be used as absorption and/or adsorption-promoting agents within the methods and compositions of the invention to enhance delivery and adsorption of one or more Y2 receptor-binding peptide proteins, analogs and mimetics, and other biologically active agent(s), including to enhance delivery of the active agent(s) to a target tissue or compartment in the subject (e.g., the liver, hepatic portal vein, CNS tissue or fluid, or blood plasma). Useful alpha, beta-olefinically unsaturated nitriles are preferably monoolefinically unsaturated nitriles having from 3 to 10 carbon atoms such as acrylonitrile, methacrylonitrile, and the like. Most preferred are acrylonitrile and methacrylonitrile. Acrylic amides containing from 3 to 35 carbon atoms including monoolefinically unsaturated amides also may be used. Representative amides include acrylamide, methacrylamide, N-t-butyl acrylamide, N-cyclohexyl acrylamide, higher alkyl amides, where the alkyl group on the nitrogen contains from 8 to 32 carbon atoms, acrylic amides including N-alkylol amides of alpha, beta-olefinically unsaturated carboxylic acids including those having from 4 to 10 carbon atoms such as N-methylol acrylamide, N-propanol acrylamide, N-methylol methacrylamide, N-methylol malcimide, N-methylol malcamic acid esters, N-methylol-p-vinyl benzamide, and the like.

Yet additional useful absorption and/or adsorption promoting materials are alpha-olefins containing from 2 to 18 carbon atoms, more preferably from 2 to 8 carbon atoms; dienes containing from 4 to 10 carbon atoms; vinyl esters and allyl esters such as vinyl acetate; vinyl aromatics such as styrene, methyl styrene and chloro-styrene; vinyl and allyl ethers and ketones such as vinyl methyl ether and methyl vinyl ketone; chloroacrylates; cyanoalkyl acrylates such as alpha-cyanomethyl acrylate, and the alpha-, beta-, and gamma-cyanopropyl acrylates; alkoxyacrylates such as methoxy ethyl acrylate; haloacrylates as chloroethyl acrylate; vinyl halides and vinyl chloride, vinylidene chloride and the like; divinyls, diacrylates and other polyfunctional monomers such as divinyl ether, diethylene glycol diacrylate, ethylene glycol dimethacrylate, methylene-bis-acrylamide, allylpentaerythritol, and the like; and bis (beta-haloalkyl) alkenyl phosphonates such as bis (beta-chloroethyl) vinyl phosphonate and the like as are known to those skilled in the art. Copolymers wherein the carboxy containing monomer is a minor constituent, and the other vinylidene monomers present as major components are readily prepared in accordance with the methods disclosed herein.

When hydrogels are employed as absorption and/or adsorption promoting agents within the invention, these may be composed of synthetic copolymers from the group of acrylic and methacrylic acids, acrylamide, methacrylamide, hydroxyethylacrylate (HEA) or methacrylate (HEMA), and vinylpyrrolidones which are water interactive and swellable. Specific illustrative examples of useful polymers, especially for the delivery of peptides or proteins, are the following types of polymers: (meth)acrylamide and 0.1 to 99 wt. % (meth)acrylic acid; (meth)acrylamides and 0.1 75 wt % (meth)acryloxyethyl trimethyammonium chloride;

(meth)acrylamide and 0.1 75 wt % (meth)acrylamide; acrylic acid and 0.1 75 wt % alkyl (meth)acrylates; (meth)acrylamide and 0.1 75 wt % AMPS.RTM. (trademark of Lubrizol Corp.); (meth)acrylamide and 0 to 30 wt % alkyl (meth)acrylamides and 0.1 75 wt % AMPS.RTM.; (meth)acrylamide and 0.1 99 wt. % HEMA; (meth)acrylamide and 0.1 to 75 wt % HEMA and 0.1 to 99% (meth)acrylic acid; (meth)acrylic acid and 0.1 99 wt % HEMA; 50 mole % vinyl ether and 50 mole % maleic anhydride; (meth)acrylamide and 0.1 to 75 wt % (meth)acryloxyalky dimethyl benzylammonium chloride; (meth)acrylamide and 0.1 to 99 wt % vinyl pyrrolidone; (meth)acrylamide and 50 wt % vinyl pyrrolidone and 0.1 99.9 wt % (meth)acrylic acid; (meth)acrylic acid and 0.1 to 75 wt % AMPS.RTM. and 0.1 75 wt % alkyl (meth)acrylamide. In the above examples, alkyl means C₁ to C₃₀, preferably C₁ to C₂₂, linear and branched and C₄ to C₁₆ cyclic; where (meth) is used, it means that the monomers with and without the methyl group are included. Other very useful hydrogel polymers are swellable, but insoluble versions of poly (vinyl pyrrolidone) starch, carboxymethyl cellulose and polyvinyl alcohol.

Additional polymeric hydrogel materials useful within the invention include (poly) hydroxyalkyl (meth)acrylate: anionic and cationic hydrogels: poly (electrolyte) complexes; poly (vinyl alcohols) having a low acetate residual: a swellable mixture of crosslinked agar and crosslinked carboxymethyl cellulose: a swellable composition comprising methyl cellulose mixed with a sparingly crosslinked agar; a water swellable copolymer produced by a dispersion of finely divided copolymer of maleic anhydride with styrene, ethylene, propylene, or isobutylene; a water swellable polymer of N-vinyl lactams; swellable sodium salts of carboxymethyl cellulose; and the like.

Other gelable, fluid imbibing and retaining polymers useful for forming the hydrophilic hydrogel for mucosal delivery of biologically active agents within the invention include pectin; polysaccharides such as agar, acacia, karaya, tragaconth, algins and guar and their crosslinked versions; acrylic acid polymers, copolymers and salt derivatives, polyacrylamides; water swellable indene maleic anhydride polymers; starch graft copolymers; acrylate type polymers and copolymers with water absorbability of about 2 to 400 times its original weight; diesters of polyglucan; a mixture of crosslinked poly (vinyl alcohol) and poly (N-vinyl-2-pyrrolidone); polyoxybutylene-polyethylene block copolymer gels; carob gum; polyester gels; poly urea gels; polyether gels; polyamide gels; polyimide gels; polypeptide gels; polyamino acid gels; poly cellulosic gels; crosslinked indene-maleic anhydride acrylate polymers; and polysaccharides.

Synthetic hydrogel polymers for use within the invention may be made by an infinite combination of several monomers in several ratios. The hydrogel can be crosslinked and generally possesses the ability to imbibe and absorb fluid and swell or expand to an enlarged equilibrium state. The hydrogel typically swells or expands upon delivery to the nasal mucosal surface, absorbing about 2 5, 5 10, 10 50, up to 50 100 or more times fold its weight of water. The optimum degree of swellability for a given hydrogel will be determined for different biologically active agents depending upon such factors as molecular weight, size, solubility and diffusion characteristics of the active agent carried by or entrapped or encapsulated within the polymer, and the specific spacing and cooperative chain motion associated with each individual polymer.

Hydrophilic polymers useful within the invention are water insoluble but water swellable. Such water-swollen polymers as typically referred to as hydrogels or gels. Such gels may be conveniently produced from water-soluble polymer by the process of crosslinking the polymers by a suitable crosslinking agent. However, stable hydrogels may also be formed from specific polymers under defined conditions of pH, temperature and/or ionic concentration, according to know methods in the art. Typically the polymers are cross-linked, that is, cross-linked to the extent that the polymers possess good hydrophilic properties, have improved physical integrity (as compared to non cross-linked polymers of the same or similar type) and exhibit improved ability to retain within the gel network both the biologically active agent of interest and additional compounds for coadministration therewith such as a cytokine or enzyme inhibitor, while retaining the ability to release the active agent(s) at the appropriate location and time.

Generally hydrogel polymers for use within the invention are crosslinked with a difunctional cross-linking in the amount of from 0.01 to 25 weight percent, based on the weight of the monomers forming the copolymer, and more preferably from 0.1 to 20 weight percent and more often from 0.1 to 15 weight percent of the crosslinking agent. Another useful amount of a crosslinking agent is 0.1 to 10 weight percent. Tri, tetra or higher multifunctional crosslinking agents may also be employed. When such reagents are utilized, lower amounts may be required to attain equivalent crosslinking density, i.e., the degree of crosslinking, or network properties that are sufficient to contain effectively the biologically active agent(s).

The crosslinks can be covalent, ionic or hydrogen bonds with the polymer possessing the ability to swell in the presence of water containing fluids. Such crosslinkers and crosslinking reactions are known to those skilled in the art and in many cases are dependent upon the polymer system. Thus a crosslinked network may be formed by free radical copolymerization of unsaturated monomers. Polymeric hydrogels may also be formed by crosslinking preformed polymers by reacting functional groups found on the polymers such as alcohols, acids, amines with such groups as glyoxal, formaldehyde or glutaraldehyde, bis anhydrides and the like.

The polymers also may be cross-linked with any polyenc, e.g. decadiene or trivinyl cyclohexane; acrylamides, such as N,N-methylene-bis (acrylamide); polyfunctional acrylates, such as trimethylol propane triacrylate; or polyfunctional vinylidene monomer containing at least 2 terminal CH₂<groups, including, for example, divinyl benzene, divinyl naphthlene, allyl acrylates and the like. In certain embodiments, cross-linking monomers for use in preparing the copolymers are polyalkenyl polyethers having more than one alkenyl ether grouping per molecule, which may optionally possess alkenyl groups in which an olefinic double bond is present attached to a terminal methylene grouping (e.g., made by the etherification of a polyhydric alcohol containing at least 2 carbon atoms and at least 2 hydroxyl groups). Compounds of this class may be produced by reacting an alkenyl halide, such as allyl chloride or allyl bromide, with a strongly alkaline aqueous solution of one or more polyhydric alcohols. The product may be a complex mixture of polyethers with varying numbers of ether groups. Efficiency of the polyether cross-linking agent increases with the number of potentially polymerizable groups on the molecule. Typically, polyethers containing an average of two or more alkenyl ether groupings per molecule are used. Other cross-linking monomers include for example, diallyl esters, dimethallyl ethers, allyl or methallyl acrylates and acrylamides, tetravinyl silane, polyalkenyl methanes, diacrylates, and dimethacrylates, divinyl compounds such as divinyl benzene, polyallyl phosphate, diallyloxy compounds and phosphite esters and the like. Typical agents are allyl pentaerythritol, allyl sucrose, trimethylolpropane triacrylate, 1,6-hexanediol diacrylate, trimethylolpropane diallyl ether, pentaerythritol triacrylate, tetramethylene dimethacrylate, ethylene diacrylate, ethylene dimethacrylate, triethylene glycol dimethacrylate, and the like. Allyl pentaerythritol, trimethylolpropane diallylether and allyl sucrose provide suitable polymers. When the cross-linking agent is present, the polymeric mixtures usually contain between about 0.01 to 20 weight percent, e.g., 1%, 5%, or 10% or more by weight of cross-linking monomer based on the total of carboxylic acid monomer, plus other monomers.

In more detailed aspects of the invention, mucosal delivery of biologically active agents disclosed herein, is enhanced by retaining the active agent(s) in a slow-release or enzymatically or physiologically protective carrier or vehicle, for example a hydrogel that shields the active agent from the action of the degradative enzymes. In certain embodiments, the active agent is bound by chemical means to the carrier or vehicle, to which may also be admixed or bound additional agents such as enzyme inhibitors, cytokines, etc. The active agent may alternately be immobilized through sufficient physical entrapment within the carrier or vehicle, e.g., a polymer matrix.

Polymers such as hydrogels useful within the invention may incorporate functional linked agents such as glycosides chemically incorporated into the polymer for enhancing intranasal bioavailability of active agents formulated therewith. Examples of such glycosides are glucosides, fructosides, galactosides, arabinosides, mannosides and their alkyl substituted derivatives and natural glycosides such as arbutin, phlorizin, amygdalin, digitonin, saponin, and indican. There are several ways in which a typical glycoside may be bound to a polymer. For example, the hydrogen of the hydroxyl groups of a glycoside or other similar carbohydrate may be replaced by the alkyl group from a hydrogel polymer to form an ether. Also, the hydroxyl groups of the glycosides may be reacted to esterify the carboxyl groups of a polymeric hydrogel to form polymeric esters in situ. Another approach is to employ condensation of acetobromoglucose with cholest-5-en-3beta-ol on a copolymer of maleic acid. N-substituted polyacrylamides can be synthesized by the reaction of activated polymers with omega-aminoalkylglycosides: (1) (carbohydrate-spacer) (n)-polyacrylamide, ‘pseudopolysaccharides “; (2) (carbohydrate spacer) (n)-phosphatidylethanolamine (m)-polyacrylamide, neoglycolipids, derivatives of phosphatidylethanolamine; (3) (carbohydrate-spacer) (n)-biotin (m)-polyacrylamide. These biotinylated derivatives may attach to lectins on the mucosal surface to facilitate absorption and/or adsorption of the biologically active agent(s), e.g., a polymer-encapsulated Y2 receptor-binding peptide.

Within more detailed aspects of the invention, one or more biologically active agents (e.g., a polyanionic electrolyte or an antioxidant), disclosed herein, optionally including secondary active agents such as protease inhibitor(s), cytokine(s), additional modulator(s) of intercellular junctional physiology, etc., are modified and bound to a polymeric carrier or matrix. For example, this may be accomplished by chemically binding a peptide or protein active agent and other optional agent(s) within a crosslinked polymer network. It is also possible to chemically modify the polymer separately with an interactive agent such as a glycosidal containing molecule. In certain aspects, the biologically active agent(s), and optional secondary active agent(s), may be functionalized, i.e., wherein an appropriate reactive group is identified or is chemically added to the active agent(s). Most often an ethylenic polymerizable group is added, and the functionalized active agent is then copolymerized with monomers and a crosslinking agent using a standard polymerization method such as solution polymerization (usually in water), emulsion, suspension, or dispersion polymerization. Often, the functionalizing agent is provided with a high enough concentration of functional or polymerizable groups to ensure that several sites on the active agent(s) are functionalized. For example, in a polypeptide comprising 16 amine sites, it is generally desired to functionalize at least 2, 4, 5, 7, and up to 8 or more of the sites.

After functionalization, the functionalized active agent(s) is/are mixed with monomers and a crosslinking agent that comprise the reagents from which the polymer of interest is formed. Polymerization is then induced in this medium to create a polymer containing the bound active agent(s). The polymer is then washed with water or other appropriate solvents and otherwise purified to remove trace unreacted impurities and, if necessary, ground or broken up by physical means such as by stirring, forcing it through a mesh, ultrasonication or other suitable means to a desired particle size. The solvent, usually water, is then removed in such a manner as to not denature or otherwise degrade the active agent(s). One desired method is lyophilization (freeze drying) but other methods are available and may be used (e.g., vacuum drying, air drying, spray drying, etc.).

To introduce polymerizable groups in peptides, proteins, and other active agents within the invention, it is possible to react available amino, hydroxyl, thiol and other reactive groups with electrophiles containing unsaturated groups. For example, unsaturated monomers containing N-hydroxy succinimidyl groups, active carbonates such as p-nitrophenyl carbonate, trichlorophenyl carbonates, tresylates, oxycarbonylimidazoles, epoxides, isocyanates, aldehydes, and unsaturated carboxymethyl azides and unsaturated orthopyridyl-disulfides belong to this category of reagents. Illustrative examples of unsaturated reagents are allyl glycidyl ether, allyl chloride, allylbromide, allyl iodide, acryloyl chloride, allyl isocyanate, allylsulfonyl chloride, maleic anhydride, copolymers of maleic anhydride and allyl ether, and the like.

All of the lysine active derivatives, except aldehyde, can generally react with other amino acids such as imidazole groups of histidine and hydroxyl groups of tyrosine and the thiol groups of cystine if the local environment enhances nucleophilicity of these groups. Aldehyde containing functionalizing reagents are specific to lysine. These types of reactions with available groups from lysines, cysteines, tyrosine have been extensively documented in the literature and are known to those skilled in the art.

In the case of biologically active agents that contain amine groups, it is convenient to react such groups with an acyloyl chloride, such as acryloyl chloride, and introduce the polymerizable acrylic group onto the reacted agent. Then during preparation of the polymer, such as during the crosslinking of the copolymer of acrylamide and acrylic acid, the functionalized active agent, through the acrylic groups, is attached to the polymer and becomes bound thereto.

In additional aspects of the invention, biologically active agents, including peptides, proteins, nucleosides, and other molecules which are bioactive in vivo, are conjugation-stabilized by covalently bonding one or more active agent(s) to a polymer incorporating as an integral part thereof both a hydrophilic moiety, e.g., a linear polyalkylene glycol, a lipophilic moiety (see, e.g., U.S. Pat. No. 5,681,811). In one aspect, a biologically active agent is covalently coupled with a polymer comprising (i) a linear polyalkylene glycol moiety and (ii) a lipophilic moiety, wherein the active agent, linear polyalkylene glycol moiety, and the lipophilic moiety are conformationally arranged in relation to one another such that the active therapeutic agent has an enhanced in vivo resistance to enzymatic degradation (i.e., relative to its stability under similar conditions in an unconjugated form devoid of the polymer coupled thereto). In another aspect, the conjugation-stabilized formulation has a three-dimensional conformation comprising the biologically active agent covalently coupled with a polysorbate complex comprising (i) a linear polyalkylene glycol moiety and (ii) a lipophilic moiety, wherein the active agent, the linear polyalkylene glycol moiety and the lipophilic moiety are conformationally arranged in relation to one another such that (a) the lipophilic moiety is exteriorly available in the three-dimensional conformation, and (b) the active agent in the composition has an enhanced in vivo resistance to enzymatic degradation.

In a further related aspect, a multiligand conjugated complex is provided which comprises a biologically active agent covalently coupled with a triglyceride backbone moiety through a polyalkylene glycol spacer group bonded at a carbon atom of the triglyceride backbone moiety, and at least one fatty acid moiety covalently attached either directly to a carbon atom of the triglyceride backbone moiety or covalently joined through a polyalkylene glycol spacer moiety (see, e.g., U.S. Pat. No. 5,681,811). In such a multiligand conjugated therapeutic agent complex, the alpha and beta carbon atoms of the triglyceride bioactive moiety may have fatty acid moieties attached by covalently bonding either directly thereto, or indirectly covalently bonded thereto through polyalkylene glycol spacer moieties. Alternatively, a fatty acid moiety may be covalently attached either directly or through a polyalkylene glycol spacer moiety to the alpha and alpha carbons of the triglyceride backbone moiety, with the bioactive therapeutic agent being covalently coupled with the gamma-carbon of the triglyceride backbone moiety, cither being directly covalently bonded thereto or indirectly bonded thereto through a polyalkylene spacer moiety. It will be recognized that a wide variety of structural, compositional, and conformational forms are possible for the multiligand conjugated therapeutic agent complex comprising the triglyceride backbone moiety, within the scope of the invention. It is further noted that in such a multiligand conjugated therapeutic agent complex, the biologically active agent(s) may advantageously be covalently coupled with the triglyceride modified backbone moiety through alkyl spacer groups, or alternatively other acceptable spacer groups, within the scope of the invention. As used in such context, acceptability of the spacer group refers to steric, compositional, and end use application specific acceptability characteristics.

In yet additional aspects of the invention, a conjugation-stabilized complex is provided which comprises a polysorbate complex comprising a polysorbate moiety including a triglyceride backbone having covalently coupled to alpha, alpha and beta carbon atoms thereof functionalizing groups including (i) a fatty acid group; and (ii) a polyethylene glycol group having a biologically active agent or moiety covalently bonded thereto, e.g., bonded to an appropriate functionality of the polyethylene glycol group. Such covalent bonding may be either direct, e.g., to a hydroxy terminal functionality of the polyethylene glycol group, or alternatively, the covalent bonding may be indirect, e.g., by reactively capping the hydroxy terminus of the polyethylene glycol group with a terminal carboxy functionality spacer group, so that the resulting capped polyethylene glycol group has a terminal carboxy functionality to which the biologically active agent or moiety may be covalently bonded.

In yet additional aspects of the invention, a stable, aqueously soluble, conjugation-stabilized complex is provided which comprises one or more biologically active agent(s)+disclosed herein covalently coupled to a physiologically compatible polyethylene glycol (PEG) modified glycolipid moiety. In such complex, the biologically active agent(s) may be covalently coupled to the physiologically compatible PEG modified glycolipid moiety by a labile covalent bond at a free amino acid group of the active agent, wherein the labile covalent bond is scissionable in vivo by biochemical hydrolysis and/or proteolysis. The physiologically compatible PEG modified glycolipid moiety may advantageously comprise a polysorbate polymer, e.g., a polysorbate polymer comprising fatty acid ester groups selected from the group consisting of monopalmitate, dipalmitate, monolaurate, dilaurate, trilaurate, monolcate, dioleate, triolcate, monostearate, distearate, and tristearate. In such complex, the physiologically compatible PEG modified glycolipid moiety may suitably comprise a polymer selected from the group consisting of polyethylene glycol ethers of fatty acids, and polyethylene glycol esters of fatty acids, wherein the fatty acids for example comprise a fatty acid selected from the group consisting of lauric, palmitic, oleic, and stearic acids.

Compositions according to the present invention are often administered in an aqueous solution as a nasal or pulmonary spray and may be dispensed in spray form by a variety of methods known to those skilled in the art. Preferred systems for dispensing liquids as a nasal spray are disclosed in U.S. Pat. No. 4,511,069. The formulations may be presented in multi-dose containers, for example in the sealed dispensing system disclosed in U.S. Pat. No. 4,511,069. Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or a mixture thereof.

Nasal and pulmonary spray solutions of the present invention typically comprise the drug or drug to be delivered, optionally formulated with a surface-active agent, such as a nonionic surfactant (e.g., polysorbate-80), and one or more buffers. In some embodiments of the present invention, the nasal spray solution further comprises a propellant. The pH of the nasal spray solution is optionally between about pH 3.0 and 6.0, preferably 5.0.+−.0.3. Suitable buffers for use within these compositions are as described above or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, chlorobutanol, benzylalkonimum chloride, and the like. Suitable surfactants include, but are not limited to, oleic acid, sorbitan trioleate, polysorbates, lecithin, phosphotidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

Within alternate embodiments, mucosal formulations are administered as dry powder formulations comprising the biologically active agent in a dry, usually lyophilized, form of an appropriate particle size, or within an appropriate particle size range, for intranasal delivery. Minimum particle size appropriate for deposition within the nasal or pulmonary passages is often about 0.5 μ mass median equivalent aerodynamic diameter (MMEAD), commonly about 1μ MMEAD, and more typically about 2μ MMEAD. Maximum particle size appropriate for deposition within the nasal passages is often about 10μ MMEAD, commonly about 8μ MMEAD, and more typically about 4μ MMEAD. Intranasally respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI), which rely on the patient's breath, upon pulmonary or nasal inhalation, to disperse the power into an aerosolized amount. Alternatively, the dry powder may be administered via air-assisted devices that use an external power source to disperse the powder into an aerosolized amount, e.g., a piston pump.

Dry powder devices typically require a powder mass in the range from about 1 mg to 20 mg to produce a single aerosolized dose (“puff”). If the required or desired dose of the biologically active agent is lower than this amount, the powdered active agent will typically be combined with a pharmaceutical dry bulking powder to provide the required total powder mass. Preferred dry bulking powders include sucrose, lactose, dextrose, mannitol, glycine, trehalose, human serum albumin (HSA), and starch. Other suitable dry bulking powders include cellobiose, dextrans, maltotriose, pectin, sodium citrate, sodium ascorbate, and the like.

To formulate compositions for mucosal delivery within the present invention, the biologically active agent can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about ⅓ to 3, more typically ½ to 2, and most often ¾ to 1.7.

The compositions formulated for inhalation via nebulization may have a comprise droplets of the composition. A composition of droplets may have a distribution of droplet sizes from 0.1 μm 50 μm. A composition of droplets may have a distribution of droplet sizes from 1 μm 30 μm. A composition of droplets may have a distribution of droplet sizes from 1 μm 20 μm. In some embodiments, the nebulized compositions may comprise an average droplet diameter of 1 μm to 30 μm, 1 μm to 20 μm, 1 μm to 12 μm, 1 μm to 10 μm, 1 μm to 8 μm, 1 μm to 5 μm, 2 μm to 4 μm, or 2 μm to 3 μm.

### Methods

A method of the present disclosure may comprise administering one or more compositions of the present disclosure (e.g., a composition comprising heparin, N-acetylcysteine, or both) to a subject in need thereof. In some embodiments, the subject may be undergoing or have recently undergone chemotherapy or radiation therapy for treatment of a cancer (e.g., a lung cancer). Administration of the composition may treat pulmonary damage (e.g., pulmonary damage associated with radiation therapy, chemotherapy, or both) in the subject. In some embodiments, a method of the present disclosure may comprise administering one or more active agents (e.g., heparin, N-acetylcysteine, or both) to a subject in need thereof. The subject may be a human subject. The subject may have or be at risk of developing radiation-induced or chemotherapy-induced pulmonary damage. In some embodiments, a method to treat pulmonary damage (e.g., pulmonary damage associated with radiation therapy, chemotherapy, or both) may comprise administering one or more active agents. An active agent may be a polyanionic electrolyte (e.g., heparin) or an antioxidant (e.g., N-acetylcysteine). In some embodiments, the one or more active agents may be administered in a single composition. In some embodiments, one or more active agents may be administered separately.

### Dosing Regiments

In some embodiments, a method of the present disclosure may comprise administering a dose of a heparin, and optionally a dose of N-acetylcysteine, by inhalation via nebulization at regular intervals over a treatment duration. For example, heparin (e.g., a low molecular weight heparin such as enoxaparin) may be administered to a patient having or at risk of developing pulmonary damage (e.g., pulmonary damage associated with radiation therapy, chemotherapy, or both) about every 12 hours for about 7 days, or until symptoms of the pulmonary damage improve. In another example, heparin (e.g., a low molecular weight heparin such as enoxaparin) may be administered to a patient having or at risk of developing pulmonary damage about every 12 hours followed along with N-acetylcysteine administered about 30 minutes after each dose of heparin. In some embodiments, alternating doses of N-acetylcysteine may be administered concurrently with heparin. In some embodiments, a heparin, N-acetylcysteine, or both may be administered to a patient having or at risk of developing pulmonary damage about 2 times per day. In some embodiments, the heparin, the N-acetylcysteine or both may be administered for about 14 days followed by 14 days without heparin treatment. An administration cycle (e.g., 7 days of treatment followed by 7 days of no treatment) may be repeated. In some embodiments, the N-acetylcysteine may be administered at from about 500 mg per dose to about 700 mg per dose. In some embodiments, the heparin may be administered at from about 0.5 mg/kg per dose to about 2 mg/kg per dose. In some embodiments, the heparin may be administered at from about 30 mg to about 200 mg per dose for an adult or from about 10 mg to about 100 mg per dose for a child. In some embodiments, the heparin may be administered at from about 50 IU/kg per dose to about 200 IU/kg per dose. International standard units (IU) of heparin may the anti-Factor Xa activity of heparin. Anti-factor Xa activity of heparin may be based on reference to the W.H.O. First International Low Molecular Weight Heparin Reference Standard.

In some embodiments, heparin (e.g., a low molecular weight heparin) may be administered to a patient having or at risk of developing pulmonary damage about every 6, about every 7, about every 8, about every 9, about every 10, about every 11, about every 12, about every 13, about every 14, about every 15, about every 16, about every 17, or about every 18 hours for a desired treatment duration. In some embodiments, heparin may be administered to a patient having or at risk of developing pulmonary damage about every 3 to 5 hours, every 4 to 6 hours, every 5 to 7 hours, every 6 to 8 hours, every 7 to 9 hours, every 8 to 10 hours, every 9 to 11 hours, every 10 to 12 hours, every 11 to 13 hours, every 12 to 14 hours, every 13 to 15 hours, every 14 to 16 hours, every 15 to 17 hours, every 16 to 18 hours, every 17 to 19 hours, every 19 to 21 hours, every 20 to 22 hours, every 21 to 23 hours, or every 22 to 24 hours for a desired treatment duration. In some embodiments, N-acetylcysteine may be administered to a patient having or at risk of developing pulmonary damage about every 1, about every 2, about every 3, about every 4, about every 5, about every 6, about every 7, about every 8, about every 9, about every 10, about every 11, about every 12, about every 13, about every 14, about every 15, about every 16, about every 17, or about every 18 hours. In some embodiments, heparin may be administered about 1, 2, 3, 4, 5, 6, 7, or 8 times per day. In some embodiments, N-acetylcysteine may be administered about 1, 2, 3, 4, 5, 6, 7, 8, 9, or 10 times per day.

In some embodiments, a treatment comprising heparin, and optionally N-acetylcysteine, may be administered for a treatment duration of about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, or about 21 days. In some embodiments, a treatment comprising heparin, and optionally N-acetylcysteine, may be administered for a treatment duration of up to about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, or about 28 days. In some embodiments, a treatment comprising heparin, and optionally N-acetylcysteine, may be administered until symptoms of the lung damage improve.

In some embodiments, heparin may be administered at about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.2 mg/kg, about 1.4 mg/kg, about 1.6 mg/kg, about 1.8 mg/kg, about 2 mg/kg, about 2.2 mg/kg, about 2.4 mg/kg, about 2.6 mg/kg, about 2.8 mg/kg, about 3 mg/kg, about 3.5 mg/kg, about 4 mg/kg, about 4.5 mg/kg, about 5 mg/kg, about 5.5 mg/kg, or about 6 mg/kg per dose. In some embodiments, heparin may be administered at from about 0.1 mg/kg to about 1 mg/kg, from about 0.1 mg/kg to about 2 mg/kg, from about 0.1 mg/kg to about 3 mg/kg, from about 0.1 mg/kg to about 4 mg/kg, from about 0.1 mg/kg to about 5 mg/kg, from about 0.1 mg/kg to about 6 mg/kg, from about 0.5 mg/kg to about 1 mg/kg, from about 0.5 mg/kg to about 2 mg/kg, from about 0.5 mg/kg to about 3 mg/kg, from about 0.5 mg/kg to about 4 mg/kg, from about 0.5 mg/kg to about 5 mg/kg, from about 0.5 mg/kg to about 6 mg/kg, from about 1 mg/kg to about 2 mg/kg, from about 1 mg/kg to about 3 mg/kg, from about 1 mg/kg to about 4 mg/kg, from about 1 mg/kg to about 5 mg/kg, from about 1 mg/kg to about 6 mg/kg, from about 2 mg/kg to about 3 mg/kg, from about 2 mg/kg to about 4 mg/kg, from about 2 mg/kg to about 5 mg/kg, from about 2 mg/kg to about 6 mg/kg, from about 3 mg/kg to about 4 mg/kg, from about 3 mg/kg to about 5 mg/kg, from about 3 mg/kg to about 6 mg/kg, from about 4 mg/kg to about 5 mg/kg, from about 4 mg/kg to about 6 mg/kg, or from about 5 mg/kg to about 6 mg/kg per dose.

In some embodiments, heparin may be administered at about 10 IU/kg, about 20 IU/kg, about 30 IU/kg, about 40 IU/kg, about 50 IU/kg, about 60 IU/kg, about 70 IU/kg, about 80 IU/kg, about 90 IU/kg, about 100 IU/kg, about 120 IU/kg, about 140 IU/kg, about 160 IU/kg, about 180 IU/kg, about 200 IU/kg, about 220 IU/kg, about 240 IU/kg, about 260 IU/kg, about 280 IU/kg, about 300 IU/kg, about 350 IU/kg, about 400 IU/kg, about 450 IU/kg, about 500 IU/kg, about 550 IU/kg, or about 600 IU/kg per dose. In some embodiments, heparin may be administered at from about 10 IU/kg to about 100 IU/kg, from about 10 IU/kg to about 200 IU/kg, from about 10 IU/kg to about 300 IU/kg, from about 10 IU/kg to about 400 IU/kg, from about 10 IU/kg to about 500 IU/kg, from about 10 IU/kg to about 600 IU/kg, from about 50 IU/kg to about 100 IU/kg, from about 50 IU/kg to about 200 IU/kg, from about 50 IU/kg to about 300 IU/kg, from about 50 IU/kg to about 400 IU/kg, from about 50 IU/kg to about 500 IU/kg, from about 50 IU/kg to about 600 IU/kg, from about 100 IU/kg to about 200 IU/kg, from about 100 IU/kg to about 300 IU/kg, from about 100 IU/kg to about 400 IU/kg, from about 100 IU/kg to about 500 IU/kg, from about 100 IU/kg to about 600 IU/kg, from about 200 IU/kg to about 300 IU/kg, from about 200 IU/kg to about 400 IU/kg, from about 200 IU/kg to about 500 IU/kg, from about 200 IU/kg to about 600 IU/kg, from about 300 IU/kg to about 400 IU/kg, from about 300 IU/kg to about 500 IU/kg, from about 300 IU/kg to about 600 IU/kg, from about 400 IU/kg to about 500 IU/kg, from about 400 IU/kg to about 600 IU/kg, or from about 500 IU/kg to about 600 IU/kg per dose. International standard units (IU) of heparin may the anti-Factor Xa activity of heparin. Anti-factor Xa activity of heparin may be based on reference to the W.H.O. First International Low Molecular Weight Heparin Reference Standard.

In some embodiments, heparin may be administered at about 10 mg, about 20 mg, about 30 mg, about 40 mg, about 50 mg, about 60 mg, about 70 mg, about 80 mg, about 90 mg, about 100 mg, about 120 mg, about 140 mg, about 160 mg, about 180 mg, about 200 mg, about 220 mg, about 240 mg, about 260 mg, about 280 mg, about 300 mg, about 350 mg, about 400 mg, about 450 mg, about 500 mg, about 550 mg, or about 600 mg per dose. In some embodiments, heparin may be administered at from about 10 mg to about 100 mg, from about 10 mg to about 200 mg, from about 10 mg to about 300 mg, from about 10 mg to about 400 mg, from about 10 mg to about 500 mg, from about 10 mg to about 600 mg, from about 50 mg to about 100 mg, from about 50 mg to about 200 mg, from about 50 mg to about 300 mg, from about 50 mg to about 400 mg, from about 50 mg to about 500 mg, from about 50 mg to about 600 mg, from about 100 mg to about 200 mg, from about 100 mg to about 300 mg, from about 100 mg to about 400 mg, from about 100 mg to about 500 mg, from about 100 mg to about 600 mg, from about 200 mg to about 300 mg, from about 200 mg to about 400 mg, from about 200 mg to about 500 mg, from about 200 mg to about 600 mg, from about 300 mg to about 400 mg, from about 300 mg to about 500 mg, from about 300 mg to about 600 mg, from about 400 mg to about 500 mg, from about 400 mg to about 600 mg, or from about 500 mg to about 600 mg per dose.

In some embodiments, N-acetylcysteine may be administered at about 20 mg, about 40 mg, about 60 mg, about 80 mg, about 100 mg, about 120 mg, about 140 mg, about 160 mg, about 180 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg per dose. In some embodiments, N-acetylcysteine may be administered at from about 20 mg to about 200 mg, from about 20 mg to about 400 mg, from about 20 mg to about 600 mg, from about 20 mg to about 800 mg, from about 50 mg to about 1000 mg, from about 50 mg to about 200 mg, from about 50 mg to about 400 mg, from about 50 mg to about 600 mg, from about 50 mg to about 800 mg, from about 50 mg to about 1000 mg, from about 100 mg to about 200 mg, from about 100 mg to about 400 mg, from about 100 mg to about 600 mg, from about 100 mg to about 800 mg, from about 100 mg to about 1000 mg, from about 200 mg to about 400 mg, from about 200 mg to about 600 mg, from about 200 mg to about 800 mg, from about 200 mg to about 1000 mg, from about 400 mg to about 600 mg, from about 400 mg to about 800 mg, from about 400 mg to about 1000 mg, from about 600 mg to about 800 mg, from about 600 mg to about 1000 mg, or from about 800 mg to about 1000 mg per dose.

### Methods of Treating Lung Damage

A composition of the present disclosure (e.g., a heparin composition, an N-acetylcysteine composition, or a composition comprising heparin and N-acetylcysteine) may be used in a method of treating lung damage. In some embodiments, the lung damage is associated with a cancer therapy (e.g., radiation therapy or chemotherapy). The subject may be undergoing treatment for a cancer (e.g., a lung cancer). In some embodiments, the subject may continue receiving cancer treatment while undergoing the methods described herein (e.g., comprising administering heparin and/or N-acetylcysteine to treat lung damage). A composition described herein may be administered directly to the lungs via inhalation (e.g., via nebulization of the composition). In some embodiments, the subject has previously undergone treatment for cancer. In some embodiments, the has previously undergone treatment for cancer within the last six months. In some embodiments, the has previously undergone treatment for cancer within the last six months, eight months, ten months, or twelve months. In some embodiments, the subject has pneumonitis. In some embodiments, the subject has pulmonary fibrosis.

A method of the present disclosure may comprise administering a composition comprising a heparin (e.g., bemiparin, nadroparin, reviparin, cnoxaparin, parnaparin, certoparin, dalteparin, danaparoid, or tinzaparin). The composition may comprise from 500 IU to 1000 IU, from 1000 IU to 1500 IU, from 1500 IU to 2000 IU, from 2000 IU to 2500 IU, from 2500 IU to 3000 IU, from 3000 IU to 3500 IU, from 3500 IU to 4000 IU, from 4000 IU to 4500 IU, from 4500 IU to 5000 IU, from 5000 IU to 5500 IU, from 5500 IU to 6000 IU, from 6000 IU to 6500 IU, from 6500 IU to 7000 IU, from 7000 IU to 7500 IU, from 7500 IU to 8000 IU, from 8000 IU to 8500 IU, from 8500 IU to 9000 IU, from 9000 IU to 9500 IU, from 9500 IU to 10000 IU, from 10000 IU to 20000 IU, from 20000 IU to 30000 IU, from 30000 IU to 40000 IU, from 40000 IU to 50000 IU, from 50000 IU to 60000 IU, from 60000 IU to 70000 IU, from 70000 IU to 80000 IU, from 80000 IU to 90000 IU, from 90000 IU to 100000 IU, from 100000 IU to 110000 IU, from 110000 IU to 120000 IU, from 120000 IU to 130000 IU, from 130000 IU to 140000 IU, from 140000 IU to 150000 IU, from 150000 IU to 160000 IU, from 160000 IU to 170000 IU, from 170000 IU to 180000 IU, from 180000 IU to 190000 IU, or from 190000 IU to 200000 IU of polyanionic electrolyte. In some embodiments, from 500 IU to 1000 IU, from 1000 IU to 1500 IU, from 1500 IU to 2000 IU, from 2000 IU to 2500 IU, from 2500 IU to 3000 IU, from 3000 IU to 3500 IU, from 3500 IU to 4000 IU, from 4000 IU to 4500 IU, from 4500 IU to 5000 IU, from 5000 IU to 5500 IU, from 5500 IU to 6000 IU, from 6000 IU to 6500 IU, from 6500 IU to 7000 IU, from 7000 IU to 7500 IU, from 7500 IU to 8000 IU, from 8000 IU to 8500 IU, from 8500 IU to 9000 IU, from 9000 IU to 9500 IU, from 9500 IU to 10000 IU, from 10000 IU to 20000 IU, from 20000 IU to 30000 IU, from 30000 IU to 40000 IU, from 40000 IU to 50000 IU, from 50000 IU to 60000 IU, from 60000 IU to 70000 IU, from 70000 IU to 80000 IU, from 80000 IU to 90000 IU, from 90000 IU to 100000 IU, from 100000 IU to 110000 IU, from 110000 IU to 120000 IU, from 120000 IU to 130000 IU, from 130000 IU to 140000 IU, from 140000 IU to 150000 IU, from 150000 IU to 160000 IU, from 160000 IU to 170000 IU, from 170000 IU to 180000 IU, from 180000 IU to 190000 IU, or from 190000 IU to 200000 IU of heparin may be administered to a subject per day. In some embodiments, from 20,000 IU to 100,000 IU, or from 20,000 IU to 70,000 IU of the heparin (e.g., bemiparin, nadroparin, reviparin, enoxaparin, parnaparin, certoparin, dalteparin, or tinzaparin) may be administered to the subject per day. The terms “international standard units,” “IU,” and “units” are used interchangeably herein to refer to an amount of an active compound. International standard units (IU) of heparin may the anti-Factor Xa activity of heparin. Anti-factor Xa activity of heparin may be based on reference to the W.H.O. First International Low Molecular Weight Heparin Reference Standard.

A method of the present disclosure may comprise administering a composition comprising an antioxidant (e.g., N-acetylcysteine). The composition may comprise from 10 mg to 50 mg, from 50 mg to 100 mg, from 100 mg to 150 mg, from 150 mg to 200 mg, from 200 mg to 250 mg, from 250 mg to 300 mg, from 300 mg to 350 mg, from 350 mg to 400 mg, from 400 mg to 450 mg, from 450 mg to 500 mg, from 500 mg to 550 mg, from 550 mg to 600 mg, from 600 mg to 650 mg, from 650 mg to 700 mg, from 700 mg to 750 mg, from 750 mg to 800 mg, from 800 mg to 850 mg, from 850 mg to 900 mg, from 900 mg to 950 mg, from 950 mg to 1000 mg, from 1000 mg to 2000 mg, from 2000 mg to 3000 mg, from 3000 mg to 4000 mg, from 4000 mg to 5000 mg, or from 5000 mg to 10000 mg of antioxidant. In some embodiments, from 10 mg to 50 mg, from 50 mg to 100 mg, from 100 mg to 150 mg, from 150 mg to 200 mg, from 200 mg to 250 mg, from 250 mg to 300 mg, from 300 mg to 350 mg, from 350 mg to 400 mg, from 400 mg to 450 mg, from 450 mg to 500 mg, from 500 mg to 550 mg, from 550 mg to 600 mg, from 600 mg to 650 mg, from 650 mg to 700 mg, from 700 mg to 750 mg, from 750 mg to 800 mg, from 800 mg to 850 mg, from 850 mg to 900 mg, from 900 mg to 950 mg, from 950 mg to 1000 mg, from 1000 mg to 2000 mg, from 2000 mg to 3000 mg, from 3000 mg to 4000 mg, from 4000 mg to 5000 mg, or from 5000 mg to 10000 mg of antioxidant may be administered to a subject per day. In some embodiments, from 1 g to 30 g, from 1 g to 20 g, from 1 g to 10 g, or from 1 g to 5 g of antioxidant (e.g., N-acetylcysteine) may be administered to a subject per day.

A composition of the present disclosure may be administered 1, 2, 3, 4, 5, 6, 7, 8, 9, 10, 11, 12, 13, 14, 15, 16, 17, 18, 19, 20, 21, 22, 23, or 24 times per day. In some embodiments, a composition (e.g., a composition formulated for inhalation) may be administered over a duration of from about 1 hour to about 2 hours, from about 2 hours to about 3 hours, from about 3 hours to about 4 hours, from about 4 hours to about 5 hours, from about 5 hours to about 6 hours, from about 6 hours to about 7 hours, from about 7 hours to about 8 hours, from about 8 hours to about 9 hours, from about 9 hours to about 10 hours, from about 10 hours to about 11 hours, from about 11 hours to about 12 hours, from about 12 hours to about 13 hours, from about 13 hours to about 14 hours, from about 14 hours to about 15 hours, from about 15 hours to about 16 hours, from about 16 hours to about 17 hours, from about 17 hours to about 18 hours, from about 18 hours to about 19 hours, from about 19 hours to about 20 hours, from about 20 hours to about 21 hours, from about 21 hours to about 22 hours, from about 22 hours to about 23 hours, or from about 23 hours to about 24 hours. In some embodiments, a composition (e.g., a composition comprising heparin and N-acetylcysteine) may be administered for 1, 2, 3, 4, 5, 6, 7, 8, 9, 10, 11, 12, 13, 14, 15, 18, 21, or 28 days. In some embodiments, a composition may be administered concurrent with or following a cancer therapy (e.g., radiation therapy or chemotherapy). For example, a composition may be administered 1, 2, 3, 4, 5, 6, 7, 8, 9, or 10 weeks after receiving radiation therapy.

In this area the following definitions are useful. Aerosol is a product that is packaged under pressure and contains therapeutically active ingredients that are released upon activation of an appropriate valve system. Metered aerosol is a pressurized dosage form comprised of metered dose valves, which allow for the delivery of a uniform quantity of spray upon each activation. Powder aerosol is a product that is packaged under pressure and contains therapeutically active ingredients in the form of a powder, which are released upon activation of an appropriate valve system. Spray aerosol is an aerosol product that utilizes a compressed gas as the propellant to provide the force necessary to expel the product as a wet spray; it generally applicable to solutions of medicinal agents in aqueous solvents. Spray is a liquid minutely divided as by a jet of air or steam. Nasal spray drug products contain therapeutically active ingredients dissolved or suspended in solutions or mixtures of excipients in non-pressurized dispensers. Metered spray is a non-pressurized dosage form consisting of valves that allow the dispensing of a specified quantity of spray upon each activation. Suspension spray is a liquid preparation containing solid particles dispersed in a liquid vehicle and in the form of course droplets or as finely divided solids.

The fluid dynamic characterization of the aerosol spray emitted by metered nasal spray pumps as a drug delivery device (“DDD”). Spray characterization is an integral part of the regulatory submissions necessary for Food and Drug Administration (“FDA”) approval of research and development, quality assurance and stability testing procedures for new and existing nasal spray pumps.

Thorough characterization of the spray's geometry has been found to be the best indicator of the overall performance of nasal spray pumps. In particular, measurements of the spray's divergence angle (plume geometry) as it exits the device; the spray's cross-sectional ellipticity, uniformity and particle/droplet distribution (spray pattern); and the time evolution of the developing spray have been found to be the most representative performance quantities in the characterization of a nasal spray pump. During quality assurance and stability testing, plume geometry and spray pattern measurements are key identifiers for verifying consistency and conformity with the approved data criteria for the nasal spray pumps.

Plume Height is the measurement from the actuator tip to the point at which the plume angle becomes non-linear because of the breakdown of linear flow. Based on a visual examination of digital images, and to establish a measurement point for width that is consistent with the farthest measurement point of spray pattern, a height of 30 mm is defined for this study. Major Axis is the largest chord that can be drawn within the fitted spray pattern that crosses the COMw in base units (mm). Minor Axis is the smallest chord that can be drawn within the fitted spray pattern that crosses the COMw in base units (mm). Ellipticity Ratio is the ratio of the major axis to the minor axis D₁₀ is the diameter of droplet for which 10% of the total liquid volume of sample consists of droplets of a smaller diameter (μm). D₅₀ is the diameter of droplet for which 50% of the total liquid volume of sample consists of droplets of a smaller diameter (μm), also known as the mass median diameter. D₉₀ is the diameter of droplet for which 90% of the total liquid volume of sample consists of droplets of a smaller diameter (μm) Span—measurement of the width of the distribution. The smaller the value, the narrower the distribution. Span is calculated as: (D₉₀-D₁₀) D₅₀. % RSD is the percent relative standard deviation, the standard deviation divided by the mean of the series and multiplied by 100, also known as % CV.

A composition of the present disclosure may be administered nasally in any of the spray patterns described herein. A composition may be administered nasally in from about 0.01 mL to about 0.1 mL, from about 0.05 mL to about 0.15 mL, from about 0.1 mL to about 0.2 mL, from about 0.01 mL to about 0.3 mL, or from about 0.05 mL to about 0.5 mL per spray. A composition of the present disclosure may be administered nasally about 1, 2, 3, 4, 5, 6, 7, 8, 9, or 10 times per day. A composition may be administered nasally for about 1, 2, 3, 4, 5, 6, 7, 8, 9, 10, 11, 12, 13, 14, 15, 16, 17, 18, 19, or 20 days. The total amount of the composition administered during a course of treatment may be from about 1 mL to about 5 mL, from about 2 mL to about 6 mL, from about 3 mL to about 7 mL, from about 4 mL to about 8 mL, from about 5 mL to about 9 mL, from about 6 mL to about 10 mL, from about 7 mL to about 11 mL, from about 8 mL to about 12 mL, from about 9 mL to about 13 mL, or from about 10 mL to about 14 mL.

### Pharmaceutical Formulations

A composition of the present disclosure (comprising one or more active agents) may be formulated as a pharmaceutical composition. A pharmaceutical composition may comprise a pharmaceutically acceptable carrier or excipient. As used herein “pharmaceutically acceptable” or “pharmacologically acceptable” includes molecular entities and compositions that do not produce an adverse, allergic or other untoward reaction when administered to a subject, as appropriate. “Pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients are often also incorporated into the compositions.

A pharmaceutical composition comprising an active agent of the present disclosure is formulated according to known methods to prepare pharmaceutically useful compositions, for example, as found in “Excipient Selection in Parenteral Formulation Development” Pramanick et. al., Pharma Times, Vol. 45, No. 3, March 2013, incorporated in its entirety herein by reference. In some aspects, the active agent is combined with a pharmaceutically acceptable carrier. A composition is said to be a pharmaceutically acceptable carrier if its administration is tolerated by a recipient patient. Sterile phosphate-buffered saline is one example of a pharmaceutically acceptable carrier. Other suitable carriers are well-known to those in the art. See, for example, Gennaro (ed.), Remington's Pharmaceutical Sciences, 19th Edition (Mack Publishing Company 1995).

Formulations for administration of the active agents of the present disclosure are typically provided but are not limited to as liquid, solid or semi-solid products or dosage forms, exemplified by tablets, capsules, pellets, a powder or a lyophilized product. In some aspects, the active agent is formulated to comprise no additional materials except for a pharmaceutical carrier. In some other aspects, the active agent is formulated such that it comprises a core “matrix material” which encapsulates, binds to, coats or is adjacent to the active agent. In some other aspects, the active agent and matrix material further comprises a protective coating. Various formulations are well-known to those in the art. See, for example, Gennaro (ed.), Remington's Pharmaceutical Sciences, 19th Edition (Mack Publishing Company 1995).

Suitable excipients for use with the active agents of the present disclosure are often included in formulations for inhalation or for oral delivery. In some embodiments, a composition may be formulated for anal, parenteral, intravenous, or intrathecal delivery. More specifically, formulations which include active agents and one or more but not limited to suitable excipients, exemplified by matrix materials, binders, lubricants, glidants or disintegrates which aid in modulating the pharmacokinetic (PK) profile of administered active agents are preferred. In some aspects, compositions comprising active agents in combination with one or more suitable excipients and one or more specific product characteristics (such as dissolution or water content) which result in improved pharmacokinetic profiles of active agents in vivo. Thus, the in vivo performance of active agent's dosage forms/products included herein is based upon the composition of the excipients added during manufacturing and/or the final product characteristics generated through specific processing parameters and methods. Other excipients are well-known to those in the art. See, for example, Gennaro (ed.), Remington's Pharmaceutical Sciences, 19th Edition (Mack Publishing Company 1995).

Suitable carriers for intravenous administration include for example but are not limited to physiological saline or phosphate buffered saline (PBS), Tris, and solutions containing solubilizing agents, such as glucose, polyethylene glycol, polypropylene glycol, additional agents such as histidine, dextrose, mannitol and mixtures thereof. In some aspects, carriers for intravenous administration include a mixture of histidine and dextrose, Tris and dextrose or Tris and mannitol. Other carriers are well-known to those in the art. Sec, for example, Gennaro (ed.), Remington's Pharmaceutical Sciences, 19th Edition (Mack Publishing Company 1995).

The formulation often includes an aqueous vehicle. Aqueous vehicles include, by way of example and without limitation, sodium chloride solution, Ringers solution, isotonic dextrose solution, sterile water solution, dextrose and lactated Ringers solution. Nonaqueous vehicles include, by way of example and without limitation, fixed oils of vegetable origin, cottonseed oil, corn oil, sesame oil and peanut oil, benzyl benzoate, castor oil, N,N-dimethylacetamide, ethanol, dehydrated ethanol, glycerin, glycerol, N-methyl-2-pyrrolidone, polyethylene glycol and any derivative thereof, propylene glycol, safflower oil and soybean oil. Other vehicles are well-known to those in the art. See, for example, Gennaro (ed.), Remington's Pharmaceutical Sciences, 19th Edition (Mack Publishing Company 1995).

In some aspects, the composition the pharmaceutically acceptable carrier comprises an osmolyte. In some aspects, the osmolyte comprises a sugar, a sugar alcohol, or a combination thereof.

In certain aspects, the composition comprises a sugar alcohol selected from sorbitol, inositol, mannitol, xylitol and glycerol, or a combination thereof. In further aspects, the sugar alcohol comprises mannitol. In certain aspects, the composition comprises from 2% to 20% (wt/vol %) mannitol. In some aspects, the composition comprises from 2% to 10% (wt/vol %) mannitol. In further aspects, the composition comprises essentially 5% (wt/vol %) mannitol.

In other aspects, the composition comprises a sugar. In certain aspects, the sugar is selected from trehalose, lactose, sucrose, glucose, galactose, maltose, mannose, fructose, dextrose, or a combination thereof. In additional aspects, the sugar is selected from trehalose, sucrose, or a combination thereof. In some aspects, the composition comprises from 1% to 40% (wt/vol %) of trehalose, sucrose, or a combination of trehalose and sucrose. In other aspects, the composition comprises from 1% to 20% (wt/vol %) of trehalose, sucrose, or a combination of trehalose and sucrose. In additional aspects, the composition comprises 2% (wt/vol %) of trehalose, sucrose, or a combination of trehalose and sucrose.

In certain aspects, the composition further comprises an osmolyte selected from glycine, carnitine, ethanolamine, their phosphates, mono sugars, or a combination thereof. In some embodiments, cationic choline can be added to a formulation as a counter ion, for example as a counterion to polyanionic heparin.

In some aspects, the present compositions are isotonic. In other aspects, the compositions are essentially isotonic. In certain aspects, the ionic strength of the composition is less than 50 mM. In other aspects, the ionic strength of the composition is less than 10 mM.

Antimicrobial agents in bacteriostatic or fungistatic concentrations are typically added to preparations packaged in multiple dose containers which include by way of example and without limitation, phenols or cresols, mercurials, benzyl alcohol, chlorobutanol, methyl and propyl p-hydroxybenzoic acid esters, thimerosal, benzalkonium chloride and benzethonium chloride. Other antimicrobial agents are well-known to those in the art. See, for example, Gennaro (ed.), Remington's Pharmaceutical Sciences, 19th Edition (Mack Publishing Company 1995).

Buffers include by way of example and without limitation, acetate, ammonium sulfate, ammonium hydroxide, arginine, aspartic acid, benzene sulfonic acid, benzoate sodium, benzoate acid, carbonate, sodium carbonate, carbon dioxide, citrate, diethanolamine, glucono delta lactone, glycine, glycine HCl, histidine, histidine HCl, hydrochloric acid, hydrobromic acid, lysine maleic acid, meglumine, methanesulfonic acid, monoethanolamine, phosphate, sodium. phosphate, citrate, succinate sodium, sulfuric acid, tartarate sodium, tromethamine, sodium citrate, hydroxide, sodium hydroxide, Tris base, Tris base-65, Tris acetate, Tris HCl, and Tris HCl-65.

In various aspects, the pharmaceutically acceptable carrier comprises a buffer. In some aspects, the buffer is selected from tris, HEPES, histidine, ethylene diamine, or a combination thereof. In other aspects, the buffer is selected from tris, histidine, or a combination thereof. In further aspects, the buffer comprises histidine, which is optionally L-histidine. In additional aspects, the composition comprises at least 100 mM histidine. In further aspects, the composition comprises at least 50 mM histidine. In some aspects, the composition comprises at least 20 mM histidine. In additional aspects, the composition comprises 10 to 100 mM histidine. In other aspects, the composition comprises 10 to 20 mM histidine.

Antioxidants include by way of example and without limitation, sodium bisulfate, acetone sodium bisulfate, argon, ascorbyl palmitate, ascorbate sodium, ascorbate acid, butylated hydroxy anisole, butylated hydroxy toluene, cysteine, cystenate HCl, dithionite sodium, gentistic acid, gentistic acid ethanoloamine, glutamate monosodium, glutathione, formaldehyde solfoxylate sodium, metabisulfite potassium, metabisulfite sodium, methionine, monothioglycerol, nitrogen, propyl gallate, sulfite sodium, tocopherol alpha, alpha tocopherol hydrogen succinate and thioglycolyate sodium.

In some aspects, the compositions comprise an antioxidant, a free radical scavenger, a quencher, an antioxidant synergist or a combination thereof.

In some aspects, the antioxidant is selected from methionine, butylated hydroxytoluene, butylated hydroxyanisole, propyl gallate, or a combination thereof. In other aspects, the antioxidant comprises methionine. In further aspects, the antioxidant is L-methionine. In certain aspects, the compositions comprise at least 20 mM methionine. In other aspects, aspects, the compositions comprise at least 10 mM methionine.

Suspending, emulsifying and/or dispersing agents include by way of example and without limitation, sodium carboxymethylcelluose, hydroxypropyl methylcellulose, Polysorbate 80 (TWEEN® 80) and polyvinylpyrrolidone.

In various aspects, the compositions comprise a surfactant. In certain aspects, the surfactant is selected from polysorbate 20, polysorbate 80, a pluronic, polyoxyethylene sorbitan mono-oleate, polyethylene mono-laureate, N-actylglucoside, or a combination thereof. In certain aspects, the surfactant is polysorbate 20. In further aspects, the compositions comprise from 0.0001% to 0.1% (wt/vol %) polysorbate 20. In additional aspects, the compositions comprise cyclodextrin. In further aspects, the cyclodextrin comprises (2-hydroxypropyl)-β-cyclodextrin.

A sequestering or chelating agent of metal ions include by way of example and without limitation, calcium disodium EDTA, disodium EDTA, sodium EDTA, calcium versetaminde sodium, calteridol and DPTA. In some aspects, the present compositions comprise a metal chelator. In certain aspects, the metal chelator is selected from EDTA, deferoxamine mesylate, EGTA, fumaric acid, and malic acid, salts thereof, or combinations thereof. In further aspects, the metal chelator comprises EDTA or salts thereof. In certain aspects, the compositions have an EDTA concentration of about 0.1 mg/ml to about 1.0 mg/ml.

In some embodiments, a composition of the present disclosure (e.g., a composition comprising N-acetylcysteine) may contain disodium edetate at a concentration of about 0.01 mg/ml to about 0.1 mg/ml, about 0.1 mg/ml to about 1.0 mg/ml, about 0.1 mg/ml to about 0.2 mg/ml, about 0.1 mg/ml to about 0.5 mg/ml, about 0.5 mg/ml to about 1.0 mg/ml, about 0.1 mg/ml to about 2.0 mg/ml, about 1.0 mg/ml to about 2.0 mg/ml, about 2.0 mg/ml to about 3.0 mg/ml, or about 3.0 mg/ml to about 5.0 mg/ml.

An example of an N-acetylcysteine formulation for inhalation may comprise acetylcysteine, disodium edetate, sodium hydroxide, and water. In some embodiments, the N-acetylcysteine may be present in the formulation at a concentration of about 20% (w/v). In some embodiments, the N-acetylcysteine may be present in the formulation at a concentration of no less than 1% and no more than 30% (w/v). In some embodiments, the N-acetylcysteine may be present in the formulation at a concentration of no less than 1% and no more than 20% (w/v). In some embodiments, the N-acetylcysteine may be present in the formulation at a concentration of no less than 1% and no more than 10% (w/v). In some embodiments, the N-acetylcysteine may be present in the formulation at a concentration of no less than 2% and no more than 10% (w/v). In some embodiments, the N-acetylcysteine may be present in the formulation at a concentration of no less than 2% and no more than 5% (w/v).

An example of a heparin formulation for inhalation may comprise heparin in an aqueous solution. In some embodiments, the composition comprises not less than 10 mg/mL and not more than 500 mg/mL of the heparin. In some embodiments, the composition comprises not less than 25 mg/mL and not more than 250 mg/mL of the heparin. In some embodiments, the composition comprises not less than 50 mg/mL and not more than 200 mg/mL of the heparin. In some embodiments, the composition comprises about 100 mg/mL of the heparin.

An example of a heparin formulation for inhalation may comprise enoxaparin sodium and water. In some embodiments, the enoxaparin sodium may be present in the formulation at a concentration of about 100 mg per 1 ml. In some embodiments, the enoxaparin sodium may be present in the formulation at a concentration of about 150 mg per 1 ml.

Other isotonic agents, buffers, antioxidants, anesthetics, suspending and dispersing agents, emulsifying agents and chelating agents are well-known to those in the art. Sec, for example, Gennaro (ed.), Remington's Pharmaceutical Sciences, 19th Edition (Mack Publishing Company 1995).

Pharmaceutical carriers also include, by way of example and without limitation, ethyl alcohol, polyethylene glycol and propylene glycol for water miscible vehicles and sodium hydroxide, hydrochloric acid, citric acid or lactic acid. Other pharmaceutical carriers are well-known to those in the art. See, for example, Gennaro (ed.), Remington's Pharmaceutical Sciences, 19th Edition (Mack Publishing Company 1995).

The active agents described herein are often formulated using a variety of parameters including by way of example and without limitation, pH, molarity, % weight/volume, % volume/volume and the like. Other factors considered in the formulation of, stability of, storage of, shipping of active agents include by way of example and without limitation, the gas environment, container material, container color, cap material, cap color, presence of additional aspects, such as antioxidants, stabilizers, photoprotective compounds, protectants, sugars, ion chelators, ion donors or the like. Any factor which serves as any one of the above factors known to one of ordinary skill in the art is often used with the active agents described herein but not limited as such.

The preparation of pharmaceutical or pharmacological compositions are known to those of skill in the art in light of the present disclosure. General techniques for formulation and administration are found in “Remington: The Science and Practice of Pharmacy, Twentieth Edition,” Lippincott Williams & Wilkins, Philadelphia, Pa. Tablets, capsules, pills, powders, granules, dragees, gels, slurries, ointments, solutions suppositories, injections, inhalants and aerosols are examples of such formulations.

The active agents are often stored at various temperatures, including by way of example and without limitation, freezing, for example at about −20° C., about −70° C., about −100° C., about −120° C., about −150° C., about −200° C. or more than about −200° C., cold storage, for example at about 10° C., about 5° C., about 4° C., about 2° C., about 0° C., about −2° C. or more than about −5° C., or any other suitable temperature such that the composition remains stable.

In some aspects, compositions comprising the compounds described herein are stored as lyophilized solids. In some aspects, the present disclosure provides methods for producing the lyophilized composition, the method comprising providing the composition; and lyophilizing the composition, thereby producing the lyophilized composition.

Using lyophilization, it is possible to store the compounds in a manner that maintains physiological or otherwise optimal pH, isotonicity and stability. Such materials include pH buffers, preservatives, tonicity adjusting agents, antioxidants, other polymers (e.g., viscosity adjusting agents or extenders) and excipients to stabilize the labile protein against the stresses of drying and storage of the dried product. Specific illustrative examples of such additives include phosphate, citrate, or borate buffers; thimerosal; sorbic acid; methyl or propyl paraben, and chlorobutanol preservatives; sodium chloride: polyvinyl alcohol, polyvinyl pyrrolidone; mannitol, dextrose, dextran, lactose, sucrose, ethylene diamine tetra-acetic acid, and the like. Suitable formulations, known in the art, (Remington's Pharmaceutical Sciences (latest edition), Mack Publishing Company, Easton, Pa.; Arakawa et al. (1990), supra; Carpenter et al. (1991), supra; and Pikal (1990), supra).

In certain aspects, the pharmaceutically acceptable carrier comprises a reconstitution stabilizer. In other aspects, the reconstitution stabilizer comprises a water-soluble polymer. In additional aspects, the water-soluble polymer is selected from a polaxamer, a polyol, a polyethylene glycol, a polyvinylalcohol, a hydroxyethyl starch, dextran, polyvinylpyrrolidine poly (acrylic acid), or a combination thereof.

The term “reconstitution stabilizer” means any excipient which is capable of preventing aggregation of a reconstituted protein in an aqueous medium. Excipients possessing the necessary characteristics for the present invention are well-known in the art and generally function by the mechanisms of charge repulsion, steric hindrance, hydrophobic binding or specific high-affinity binding to the dried protein. Exemplary excipients include various osmolytes, various salts, water soluble synthetic and natural polymers, surfactants, sulfated polysaccharides, carrier proteins, buffers and the like (Manning et al. (1989), Pharmaceutical Research, 6:903-918; and Paborji, et al. (1994), Pharmaceutical Research, 11:764-771).

The present compounds and an effective amount of the reconstitution stabilizer are admixed under conditions effective to reduce aggregation of present compounds upon reconstitution with the reconstitution medium (e.g., a solvent and optionally other components such as antibacterials). The reconstitution stabilizer may be admixed with the compounds at a suitable time before, during or after reconstitution; preferably the reconstitution stabilizer will be pre-dissolved in the reconstitution medium. The compound is reconstituted at a temperature which is above the freezing point of the reconstitution medium, but which will not degrade the compound and which will not be deleterious to the reconstitution stabilizer; preferably the temperature will be between about 2° C. to 50° C. The time taken to mix the reconstitution stabilizer and the dried compound should be for a sufficient period to prepare a suitable admixture; preferably mixing will be for between about 1 to 30 minutes. Generally, the reconstituted formulation is used soon after reconstitution.

In certain aspects, the present compositions are reconstituted from a lyophilized form. In other aspects, the present disclosure provides methods for producing the reconstituted composition, the method comprising providing a lyophilized composition; and reconstituting the composition with a solution to produce a reconstituted composition. In various aspects, the reconstituting solution comprises water. In some aspects, the reconstituting solution is selected from sterile water, physiological saline solution, glucose solution or other aqueous solvents (e.g., alcohols such as ethyl, n-propyl or isopropyl, butyl alcohol), or a combination thereof, which are capable of dissolving the dried composition and compatible with the selected administration route and which does not negatively interfere with the compound and the reconstitution stabilizers employed.

### Applications

An active agent of the present disclosure may be used for various therapeutic applications. An active agent may be administered as a pharmaceutical composition. A pharmaceutical composition of the disclosure can be a combination of any active agent described herein with other chemical components, such as carriers, stabilizers, diluents, dispersing agents, suspending agents, thickening agents, and/or excipients. The pharmaceutical composition facilitates administration of an active agent described herein to an organism. Pharmaceutical compositions can be administered in therapeutically-effective amounts as pharmaceutical compositions by various forms and routes including, for example, intravenous, subcutaneous, intramuscular, rectal, aerosol, parenteral, ophthalmic, pulmonary, transdermal, vaginal, optic, nasal, oral, inhalation, dermal, intra-articular, intrathecal, intranasal, and topical administration. A pharmaceutical composition can be administered in a local or systemic manner, for example, via injection of the active agent described herein directly into an organ, optionally in a depot.

An active agent of the disclosure can be applied directly to an organ, or an organ tissue or cells, during a surgical procedure. In some embodiments, an active agent may be applied directly to a damaged tissue (e.g., a damaged lung tissue). For example, the active agent may be administered directly to damaged lung tissue via inhalation.

In practicing the methods of treatment or use provided herein, therapeutically-effective amounts of the active agent described herein described herein are administered in pharmaceutical compositions to a subject suffering from a condition. In some instances the pharmaceutical composition will affect the physiology of the animal, such as the immune. system, inflammatory response, or other physiologic affect. In some embodiments, the subject is a mammal such as a human. A therapeutically-effective amount can vary widely depending on the severity of the disease, the age and relative health of the subject, the potency of the compounds used, and other factors.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising an active agent described herein can be manufactured, for example, by expressing the active agent in a recombinant system, purifying the active agent, lyophilizing the active agent, mixing, dissolving, granulating, dragee-making, levigating, emulsifying, encapsulating, entrapping, or compression processes. The pharmaceutical compositions can include at least one pharmaceutically acceptable carrier, diluent, or excipient and compounds described herein as free-base or pharmaceutically-acceptable salt form.

Methods for the preparation of active agents described herein include formulating the active agent described herein with one or more inert, pharmaceutically-acceptable excipients or carriers to form a solid, semi-solid, or liquid composition. Solid compositions include, for example, powders, tablets, dispersible granules, capsules, cachets, and suppositories. These compositions can also contain minor amounts of nontoxic, auxiliary substances, such as wetting or emulsifying agents, pH buffering agents, and other pharmaceutically-acceptable additives.

“Product” or “dosage form” as used herein refers to any solid, semi-solid, lyophilized, aqueous, liquid or frozen formulation or preparation used for administration. Upon administration, the rate of release of an active moiety from a product is often greatly influenced by the excipients and/or product characteristics which make up the product itself. For example, an enteric coat on a tablet is designed to separate that tablet's contents from the stomach contents to prevent, for example, degradation of the stomach which often induces gastrointestinal discomfort or injury. According to the currently accepted conventional understanding, systemic exposure of the active moiety will be relatively insensitive to the small formulation changes.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N. Y., 1980; and Pharmaceutical Dosage Forms and Drug Delivery Systems, Seventh Ed. (Lippincott Williams & Wilkins 1999), each of which is incorporated by reference in its entirety.

An active agent of the present disclosure may be administered to a patient in an effective amount. The term “effective amount,” as used herein, can refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. Compositions containing such agents or compounds can be administered for prophylactic, enhancing, and/or therapeutic treatments. An appropriate “effective” amount in any individual case can be determined using techniques, such as a dose escalation study.

The methods, compositions, and kits of this disclosure can comprise a method to prevent, treat, arrest, reverse, or ameliorate the symptoms of a condition. The treatment can comprise treating a subject (e.g., an individual, a domestic animal, a wild animal or a lab animal afflicted with a disease or condition) with an active agent of the disclosure. Active agents of the present disclosure may be administered to treat a disease in a subject. The subject can be a human. A subject can be a human; a non-human primate such as a chimpanzee, or other ape or monkey species; a farm animal such as cattle, horse, sheep, goat, swine; a domestic animal such as a rabbit, dog, and cat; a laboratory animal including a rodent, such as a rat, mouse and guinea pig, or the like. A subject can be of any age. A subject can be, for example, an elderly adult, adult, adolescent, pre-adolescent, child, toddler, infant, or fetus in utero.

Treatment can be provided to the subject before clinical onset of disease. Treatment can be provided to the subject after clinical onset of disease. Treatment can be provided to the subject after 1 day, 1 week, 6 months, 12 months, or 2 years or more after clinical onset of the disease. Treatment may be provided to the subject for more than 1 day, 1 week, 1 month, 6 months, 12 months, 2 years or more after clinical onset of disease. Treatment may be provided to the subject for less than 1 day, 1 week, 1 month, 6 months, 12 months, or 2 years after clinical onset of the disease. Treatment can also include treating a human in a clinical trial. A treatment can comprise administering to a subject a pharmaceutical composition, such as one or more of the pharmaceutical compositions described throughout the disclosure. A treatment can comprise a once daily dosing. A treatment can comprise delivering an active agent of the disclosure to a subject, either intravenously, subcutaneously, intramuscularly, by inhalation, dermally, intra-articular injection, orally, intrathecally, transdermally, intranasally, via a peritoneal route, or directly onto or into a diseased tissue, e.g., via topical, intra-articular injection route or injection route of application.

In some embodiments, the present disclosure provides a method for treating pulmonary damage, the method comprising administering to a subject in need thereof an effective amount of an active agent of the present disclosure.

In some embodiments, the present disclosure provides a method for treating pulmonary damage, the method comprising administering to a patient in need thereof an effective amount of a pharmaceutical composition comprising an active agent of the present disclosure and a pharmaceutically acceptable carrier.

As used herein, the singular forms “a,” “an” and “the” include plural referents unless the context clearly dictates otherwise.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

As used herein, the term percent “identity,” in the context of two or more nucleic acid or polypeptide sequences, may refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity may be performed using the BLAST algorithm, which is described in Altschul et al. (J. Mol. Biol. 215:403-410 (1990)). Software for performing BLAST analyses is publicly available through the National Center for Biotechnology Information.

A composition can be administered alone or in combination with other treatments, either simultaneously or sequentially dependent upon the condition to be treated.

As used herein, the term “subject” broadly refers to any animal, including but not limited to, human and non-human animals (e.g., dogs, cats, cows, horses, sheep, pigs, poultry, fish, crustaceans, etc.).

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the term “therapeutically effective amount” is an amount that is effective to ameliorate a symptom of a disease. A therapeutically effective amount can be a “prophylactically effective amount” as prophylaxis can be considered therapy.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal or lingual), car, rectal, vaginal, by injection (e.g., intravenously, subcutaneously, intratumorally, intraperitoneally, etc.) and the like.

As used herein, the term “treatment” means an approach to obtaining a beneficial or intended clinical result. The beneficial or intended clinical result can include alleviation of symptoms, a reduction in the severity of the disease, inhibiting an underlying cause of a disease or condition, steadying diseases in a non-advanced state, delaying the progress of a disease, and/or improvement or alleviation of disease conditions.

As used herein, the term “pharmaceutical composition” refers to the combination of an active ingredient with a carrier, inert or active, making the composition especially suitable for therapeutic or diagnostic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), glycerol, liquid polyethylene glycols, aprotic solvents such as dimethylsulfoxide, N-methylpyrrolidone and mixtures thereof, and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 21st Ed., Mack Publ. Co., Easton, Pa. (2005), incorporated herein by reference in its entirety.

### EXAMPLES

The invention is further illustrated by the following non-limiting examples.

### Example 1

### Purified Enoxaparin Compositions

This example describes methods for making purified enoxaparin compositions. Enoxaparin, a low molecular weight heparin, was purified using an anionic resin to remove elemental impurities. The purified enoxaparin was analyzed by inductively coupled plasma-mass spectrometry (ICP-MS) for elemental impurities, including cadmium (Cd), lead (Pb), arsenic (As), mercury (Hg), cobalt (Co), vanadium (V), nickel (Ni), thallium (TI), gold (Au), palladium (Pd), iridium (Ir), osmium (Os), rhodium (Rh), ruthenium (Ru), selenium (Se), silver (Ag), platinum (Pt), lithium (Li), antimony (Sb), barium (Ba), molybdenum (Mo), copper (Cu), tin (Sn), chromium (Cr), and other elements. The analysis results of one lot of purified enoxaparin are provided in TABLE 1. ICP-MS analysis was performed in duplicate (“Repeat 1” and “Repeat 2”) and the results from each repeat were averaged (“Mean”).

TABLE 1
Elemental Impurities in Enoxaparin
Sodium as Measured by ICP-MS

| Analyte | Repeat 1 μg/g (ppm) | Repeat 2 μg/g (ppm) | Mean μg/g (ppm) |
|---|---|---|---|
| Li | <RL | <RL | <RL |
| Be | ND | ND | ND |
| B | 1.08 | 0.905 | 0.991 |
| Na | 111000 | 114000 | 112000 |
| Mg | ND | ND | ND |
| Al | ND | ND | ND |
| Si | 8.80 | 10.4 | 9.62 |
| K | 27.9 | 29.1 | 28.5 |
| Ca | 13.3 | 12.2 | 12.8 |
| Sc | ND | ND | ND |
| Ti | <RL | <RL | <RL |
| V | ND | ND | ND |
| Cr | ND | <RL | <RL |
| Mn | <RL | <RL | <RL |
| Fe | ND | ND | ND |
| Co | <RL | <RL | <RL |
| Ni | <RL | <RL | <RL |
| Cu | <RL | <RL | <RL |
| Zn | ND | ND | ND |
| Ga | <RL | <RL | <RL |
| Ge | <RL | <RL | <RL |
| As | <RL | <RL | <RL |
| Se | <RL | ND | <RL |
| Rb | <RL | <RL | <RL |
| Sr | <RL | <RL | <RL |
| Y | <RL | <RL | <RL |
| Zr | <RL | <RL | <RL |
| Nb | <RL | <RL | <RL |
| Mo | <RL | <RL | <RL |
| Ru | ND | ND | ND |
| Rh | ND | ND | ND |
| Pd | ND | ND | ND |
| Ag | ND | ND | ND |
| Cd | <RL | ND | <RL |
| In | <RL | <RL | <RL |
| Sn | 0.716 | 0.748 | 0.732 |
| Sb | <RL | <RL | <RL |
| Te | ND | ND | ND |
| Cs | <RL | <RL | <RL |
| Ba | <RL | <RL | <RL |
| La | <RL | <RL | <RL |
| Ce | <RL | <RL | <RL |
| Pr | <RL | <RL | <RL |
| Nd | <RL | <RL | <RL |
| Sm | <RL | <RL | <RL |
| Eu | ND | ND | ND |
| Gd | <RL | <RL | <RL |
| Tb | ND | ND | ND |
| Dy | <RL | <RL | <RL |
| Ho | ND | ND | ND |
| Er | <RL | ND | <RL |
| Tm | ND | ND | ND |
| Yb | <RL | <RL | <RL |
| Lu | ND | ND | ND |
| Hf | ND | ND | ND |
| Ta | ND | ND | ND |
| W | <RL | <RL | <RL |
| Re | ND | ND | ND |
| Os | ND | ND | ND |
| Ir | ND | ND | ND |
| Pt | ND | ND | ND |
| Au | ND | ND | ND |
| Hg | ND | ND | ND |
| Tl | ND | ND | ND |
| Pb | <RL | <RL | <RL |
| Bi | <RL | <RL | <RL |
| Th | <RL | <RL | <RL |
| U | ND | ND | ND |

ppm = parts per million;
ND = Not Detected;
<RL = Below Reporting Limit

A reporting limit (RL) of 0.5 g/g was used.

An enoxaparin sample is considered sufficiently free from elemental contaminants if it contains not more than 6 μg/g Cd, not more than 10 μg/g Pb, not more than 4 μg/g As, not more than 2 μg/g Hg, not more than 6 μg/g Co, not more than 2 μg/g V, not more than 10 μg/g Ni, not more than 16 μg/g T1, not more than 2 μg/g Au, not more than 2 μg/g Pd, not more than 2 μg/g Ir, not more than 2 μg/g Os, not more than 2 μg/g Rh, not more than 2 μg/g Ru, not more than 260 μg/g Se, not more than 14 μg/g Ag, not more than 2 μg/g Pt, not more than 50 μg/g Li, not more than 40 μg/g Sb, not more than 600 μg/g Ba, not more than 20 μg/g Mo, not more than 60 μg/g Cu, not more than 120 μg/g Sn, and not more than 6 μg/g Cr.

The purified enoxaparin that was sufficiently free of elemental contaminants was dissolved in water at a concentration of 100 mg/mL and stored at a temperature of no more than 25° C.

### Example 2

### Enoxaparin Compositions Formulated for Inhalation

This example describes enoxaparin compositions formulated for inhalation. The purified enoxaparin compositions from EXAMPLE 1 are used for making compositions suitable for nebulization. A solution of 100 mg/ml of enoxaparin in water is used in a nebulization device. The performance parameters of the nebulization device are evaluated including the quantification of particle sizes delivered from the mouthpiece of the nebulization device and the metered dose delivery of the enoxaparin. The total mass of the drug released from the inhalation aerosol, the quantity of the drug collected at various location on the measurement device, the average particle size is quantified for the nebulized compositions. The nebulized composition characterized is then used for treatment of a patient.

### Example 3

### Treatment of Radiation-Induced Lung Damage Using Nebulized Enoxaparin and N-Acetylcysteine

This example describes treatment of radiation-induced lung damage using nebulized enoxaparin and N-acetylcysteine. A patient with unresectable lung cancer, such as locally advanced or metastatic stage III/oligometastatic IV non-small cell lung carcinoma, who has received radiation therapy is administered nebulized enoxaparin via inhalation. The enoxaparin is purified to remove elemental impurities, as described in EXAMPLE 1, and formulated in water at a concentration of 100 mg/mL for a nebulized composition as described in EXAMPLE 2. A dose of either 0.5 mg enoxaparin per kg patient weight (mg/kg), 1 mg/kg, or 2 mg/kg of the aqueous enoxaparin formulation is nebulized and administered to the subject via inhalation every 12 hours for 14 days, followed by 14 days of no enoxaparin treatment. N-acetylcysteine is administered by inhalation via nebulization at 600 mg per dose 30 minutes after each dose of enoxaparin. The 28-day treatment cycle begins 4-6 weeks after completion of radiation therapy, and the treatment cycle is repeated for up to 6 cycles in the absence of disease progression or unacceptable toxicity. The patient continues to receive best supportive care for the lung cancer (e.g., chemotherapy therapy) while undergoing enoxaparin treatment.

The nebulized enoxaparin therapy treats, prevents, or slows the development of radiation-induced pneumonitis in the patient, decreases airway inflammation, and prevents infection, thereby treating the radiation-induced lung damage. Enoxaparin therapy inhibits release of inflammatory cytokines, such as IL-6, that can lead to treatment failure due to excessive inflammatory lung damage. Additionally, the enoxaparin therapy may reduce the recurrence of the lung cancer by slowing cancer progression through inhibition of mutagenic proliferation, adhesion, angiogenesis, migration, and invasion of cancer cells. Furthermore, the enoxaparin therapy prevents coagulation activation and pulmonary venous thromboembolism, which are hallmarks of malignant cancer and a leading cause of death in cancer patients.

### Example 4

### Treatment of Chemotherapy-Induced Lung Damage Using Nebulized Heparin and N-Acetylcysteine

This example describes treatment of chemotherapy-induced lung damage using nebulized heparin and N-acetylcysteine. A patient with unresectable lung cancer, such as locally advanced or metastatic stage III/oligometastatic IV non-small cell lung carcinoma, who is undergoing chemotherapy therapy with bleomycin, carmustine, lomustine, busulfan, daunorubicin, doxorubicin, or idarubicin is administered nebulized enoxaparin via inhalation. The enoxaparin is purified to remove elemental impurities, as described in EXAMPLE 1, and formulated in water at a concentration of 100 mg/mL for a nebulized composition as described in EXAMPLE 2. A dose of either 0.5 mg enoxaparin per kg patient weight (mg/kg), 1 mg/kg, or 2 mg/kg of the aqueous enoxaparin formulation is nebulized and administered to the subject via inhalation every 12 hours for 14 days, followed by 14 days of no enoxaparin treatment. N-acetylcysteine is administered by inhalation via nebulization at 600 mg per dose 30 minutes after each dose of enoxaparin. The 28-day treatment cycle is repeated for up to 6 cycles in the absence of disease progression or unacceptable toxicity. The patient continues to receive best supportive care for the lung cancer (e.g., chemotherapy therapy) while undergoing enoxaparin treatment.

The nebulized enoxaparin therapy treats, prevents, or slows the development of chemotherapy-induced pneumonitis in the patient, decreases airway inflammation, and prevents infection, thereby treating the chemotherapy-induced lung damage. Enoxaparin therapy inhibits release of inflammatory cytokines, such as IL-6, that can lead to treatment failure due to excessive inflammatory lung damage. Additionally, the enoxaparin therapy may reduce the recurrence of the lung cancer by slowing cancer progression through inhibition of mutagenic proliferation, adhesion, angiogenesis, migration, and invasion of cancer cells. Furthermore, the enoxaparin therapy prevents coagulation activation and pulmonary venous thromboembolism, which are hallmarks of malignant cancer and a leading cause of death in cancer patients.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a radiation-induced lung damage in a subject in need thereof, the method comprising, administering to the subject via inhalation a nebulized composition comprising a heparin, thereby treating the radiation-induced lung damage in the subject.

2. The method of claim 1, wherein the radiation-induced lung damage is caused by radiation therapy.

3. A method of treating a chemotherapy-induced lung damage in a subject in need thereof, the method comprising, administering to the subject via inhalation a nebulized composition comprising, a heparin, thereby treating the chemotherapy-induced lung damage in the subject.

4. The method of claim 3, wherein the chemotherapy-induced lung damage is caused by administering a chemotherapy comprising bleomycin, carmustine, lomustine, busulfan, daunorubicin, doxorubicin, idarubicin, or combinations thereof.

5. A method of treating a lung damage, the method comprising administering via inhalation a nebulized composition comprising a heparin to a subject who has previously undergone cancer treatment, thereby treating the lung damage in the subject.

6. The method of any one of claims 1-5, wherein the subject has previously undergone treatment for cancer within the last six months.

7. The method of any one of claims 1-6, wherein the heparin comprises a low molecular weight heparin.

8. The method of claim 7, wherein the low molecular weight heparin comprises an average molecular weight of not less than 3 kilodaltons and not more than 8 kilodaltons.

9. The method of claim 7 or claim 8, wherein the low molecular weight heparin comprises an average molecular weight of not less than 3 kilodaltons and not more than 4 kilodaltons.

10. The method of claim 7 or claim 8, wherein the low molecular weight heparin comprises an average molecular weight of not less than 4 kilodaltons and not more than 5 kilodaltons.

11. The method of claim 7 or claim 8, wherein the low molecular weight heparin comprises an average molecular weight of not less than 5 kilodaltons and not more than 6 kilodaltons.

12. The method of claim 7 or claim 8, wherein the low molecular weight heparin comprises an average molecular weight of not less than 6 kilodaltons and not more than 7 kilodaltons.

13. The method of claim 7 or claim 8, wherein the low molecular weight heparin comprises enoxaparin.

14. The method of claim 7 or claim 8, wherein the low molecular weight heparin comprises bemiparin, nadroparin, reviparin, parnaparin, certoparin, dalteparin, tinzaparin, or a combination thereof.

15. The method of any one of claims 1-14, wherein the nebulized composition comprises not less than 10 mg/mL and not more than 500 mg/mL of the heparin.

16. The method of any one of claims 1-14, wherein the nebulized composition comprises not less than 25 mg/mL and not more than 250 mg/mL of the heparin.

17. The method of any one of claims 1-14, wherein the nebulized composition comprises not less than 50 mg/mL and not more than 200 mg/mL of the heparin.

18. The method of any one of claims 1-14, wherein the nebulized composition comprises about 100 mg/mL of the heparin.

19. The method of any one of claims 1-18, wherein the nebulized composition is aqueous.

20. The method of any one of claims 1-19, further comprising administering N-acetylcysteine to the subject via inhalation.

21. The method of claim 20, wherein the N-acetylcysteine is administered at a dose of not less than 100 mg and not more than 2000 mg per dose, or not less than 300 mg or not more than 800 mg per dose.

22. The method of claim 20 or claim 21, wherein the N-acetylcysteine is administered within about 1 hour of administering the nebulized composition.

23. The method of any one of claims 20-22, wherein the nebulized composition comprises the N-acetylcysteine.

24. The method of any one of claims 1-23, wherein the subject has cancer.

25. The method of any one of claims 1-24, wherein the subject is undergoing treatment for cancer.

26. The method of claim 24 or claim 25, wherein the treatment for the cancer comprises radiation therapy, chemotherapy, or a combination thereof.

27. The method of any one of claims 24-26, wherein the cancer comprises a thoracic cancer.

28. The method of claim 27, wherein the thoracic cancer comprises lung cancer, thymic cancer, esophageal cancer, or tracheal cancer.

29. The method of claim 28, wherein the lung cancer is non-small cell lung cancer.

30. The method of any one of claims 24-29, further comprising slowing progression of the cancer in the subject.

31. The method of any one of claims 1-30, wherein the subject has pneumonitis.

32. The method of any one of claims 1-31, wherein the subject has pulmonary fibrosis.

33. The method of any one of claims 1-32, further comprising reducing a level of an inflammatory cytokine.

34. The method of claim 33, wherein the inflammatory cytokine comprises interleukin-6 (IL-6), interleukin-3 (IL-3), interleukin-7 (IL-7), interleukin-10 (IL-10), interleukin-1 (IL-1), angiopoietin-2, INF-γ, TNF-α, TGF-β, or combinations thereof.

35. The method of any one of claims 1-34, further comprising reducing airway inflammation in the subject.

36. The method of any one of claims 1-35, further comprising reducing pulmonary fibrosis in the subject.

37. The method of any one of claims 1-36, comprising administering the nebulized composition two times per day.

38. The method of any one of claims 1-37, comprising administering the nebulized composition for 14 days.

39. The method of any one of claims 1-38, comprising administering not less than 0.5 mg per kg patient weight (mg/kg) and not more than 2 mg/kg of the heparin per dose.

40. The method of any one of claims 1-39, comprising administering not less than 1 mg/kg and not more than 4 mg/kg of the heparin per day.

41. The method of any one of claims 1-40, wherein the nebulized composition comprises not more than 25 μg gold per gram of heparin, not more than 250 μg molybdenum per gram of heparin, not more than 75 μg chromium per gram of heparin, or combinations thereof.

42. The method of any one of claims 1-41, wherein the nebulized composition comprises not more than 25 μg gold per gram of heparin, not more than 250 μg molybdenum per gram of heparin, and not more than 75 μg chromium per gram of heparin.

43. The method of any one of claims 1-42, wherein the nebulized composition comprises not more than 2.5 μg gold per gram of heparin, not more than 25 μg molybdenum per gram of heparin, not more than 7.5 μg chromium per gram of heparin, or combinations thereof.

44. The method of any one of claims 1-43, wherein the nebulized composition comprises not more than 2.5 μg gold per gram of heparin, not more than 25 μg molybdenum per gram of heparin, and not more than 7.5 μg chromium per gram of heparin.

45. The method of any one of claims 1-44, wherein the nebulized composition comprises an average droplet diameter of 1 μm to 12 μm.

46. The method of any one of claims 1-45, wherein the nebulized composition comprises an average droplet diameter of 1 μm to 10 μm.

47. The method of any one of claims 1-45, wherein the nebulized composition comprises an average droplet diameter of 1 μm to 8 μm.

48. The method of any one of claims 1-45, wherein the nebulized composition comprises an average droplet diameter of 1 μm to 5 μm.

49. The method of any one of claims 1-45, wherein the nebulized composition comprises an average droplet diameter of 2 μm to 4 μm.

50. The method of any one of claims 1-45, wherein the nebulized composition comprises an average droplet diameter of 2 μm to 3 μm.

51. A composition comprising a heparin and not more than 25 μg gold per gram of heparin,
not more than 250 μg molybdenum per gram of heparin,
not more than 75 μg chromium per gram of heparin, or
combinations thereof;
wherein the composition is formulated for inhalation.

52. The composition of claim 51, wherein the heparin comprises a low molecular weight heparin.

53. The composition of claim 52, wherein the low molecular weight heparin comprises enoxaparin.

54. The composition of claim 52 or claim 53, wherein the low molecular weight heparin comprises bemiparin, nadroparin, reviparin, parnaparin, certoparin, dalteparin, tinzaparin, or a combination thereof.

55. The composition of any one of claims 51-54, wherein the composition comprises not less than 10 mg/mL and not more than 500 mg/mL of the heparin.

56. The composition of any one of claims 51-54, wherein the composition comprises not less than 25 mg/mL and not more than 250 mg/mL of the heparin.

57. The composition of any one of claims 51-54, wherein the composition comprises not less than 50 mg/mL and not more than 200 mg/mL of the heparin.

58. The composition of any one of claims 51-54, wherein the composition comprises about 100 mg/mL of the heparin.

59. The composition of any one of claims 51-54, wherein the composition is aqueous.

60. The composition of any one of claims 51-59, further comprising N-acetylcysteine.

61. The composition of claim 60, wherein the composition comprises no less than 1% and no more than 20%, no less than 1% and no more than 10%, no less than 2% and no more than 10%, or no less than 2% and no more than 5% (w/v)N-acetylcysteine.

62. The composition of any one of claims 51-61, comprising not more than 25 μg gold per gram of heparin, not more than 250 μg molybdenum per gram of heparin, and not more than 75 μg chromium per gram of heparin.

63. The composition of any one of claims 51-61, comprising not more than 2.5 μg gold per gram of heparin, not more than 25 μg molybdenum per gram of heparin, not more than 7.5 μg chromium per gram of heparin, or combinations thereof.

64. The composition of any one of claims 51-61, comprising not more than 2.5 μg gold per gram of heparin, not more than 25 μg molybdenum per gram of heparin, and not more than 7.5 μg chromium per gram of heparin.

65. The composition of any one of claims 51-62, comprising:
not less than 0.01 and not more than 7.5 μg gold per gram of heparin,
not less than 0.01 and not more than 5 μg gold per gram of heparin,
not less than 0.01 and not more than 2.5 μg gold per gram of heparin,
not less than 0.01 and not more than 1 μg gold per gram of heparin, or
not less than 0.001 and not more than 0.5 μg gold per gram of heparin.

66. The composition of any one of claims 51-62, comprising:
not less than 1 and not more than 250 μg molybdenum per gram of heparin,
not less than 1 and not more than 150 μg molybdenum per gram of heparin,
not less than 1 and not more than 100 μg molybdenum per gram of heparin,
not less than 0.1 and not more than 25 μg molybdenum per gram of heparin, or
not less than 0.001 and not more than 0.5 μg molybdenum per gram of heparin.

67. The composition of any one of claims 51-62, comprising:
not less than 0.1 and not more than 75 μg chromium per gram of heparin,
not less than 0.1 and not more than 25 μg chromium per gram of heparin,
not less than 0.1 and not more than 10 μg chromium per gram of heparin,
not less than 0.01 and not more than 7.5 μg chromium per gram of heparin, or
not less than 0.001 and not more than 0.5 μg chromium per gram of heparin.

68. The composition of any one of claims 51-67, comprising:
not more than 5 μg arsenic per gram of heparin,
not less than 0.01 and not more than 5 μg arsenic per gram of heparin,
not less than 0.01 and not more than 4 μg arsenic per gram of heparin,
not less than 0.01 and not more than 2.5 μg arsenic per gram of heparin, or
not less than 0.001 and not more than 0.5 μg arsenic per gram of heparin.

69. The composition of any one of claims 51-68, comprising:
not more than 2.5 μg mercury per gram of heparin,
not less than 0.01 and not more than 2.5 μg mercury per gram of heparin,
not less than 0.01 and not more than 1.5 μg mercury per gram of heparin,
not less than 0.01 and not more than 0.5 μg mercury per gram of heparin, or
not less than 0.001 and not more than 0.5 μg mercury per gram of heparin.

70. The composition of any one of claims 51-69, comprising:
not more than 7.5 μg cobalt per gram of heparin,
not less than 0.01 and not more than 7.5 μg cobalt per gram of heparin,
not less than 0.01 and not more than 5 μg cobalt per gram of heparin,
not less than 0.01 and not more than 2.5 μg cobalt per gram of heparin, or
not less than 0.001 and not more than 0.5 μg cobalt per gram of heparin.

71. The composition of any one of claims 51-70, comprising:
not more than 2.5 μg vanadium per gram of heparin,
not less than 0.01 and not more than 2.5 μg vanadium per gram of heparin,
not less than 0.01 and not more than 1.5 μg vanadium per gram of heparin,
not less than 0.01 and not more than 0.5 μg vanadium per gram of heparin, or
not less than 0.001 and not more than 0.5 μg vanadium per gram of heparin.

72. The composition of any one of claims 51-71, comprising:
not more than 12.5 μg nickel per gram of heparin,
not less than 0.1 and not more than 12.5 μg nickel per gram of heparin,
not less than 0.01 and not more than 7.5 μg nickel per gram of heparin,
not less than 0.01 and not more than 5 μg nickel per gram of heparin, or
not less than 0.001 and not more than 0.5 μg nickel per gram of heparin.

73. The composition of any one of claims 51-72, comprising:
not more than 2.5 μg palladium per gram of heparin,
not less than 0.01 and not more than 2.5 μg palladium per gram of heparin,
not less than 0.01 and not more than 1.5 μg palladium per gram of heparin,
not less than 0.01 and not more than 0.5 μg palladium per gram of heparin, or
not less than 0.001 and not more than 0.5 μg palladium per gram of heparin.

74. The composition of any one of claims 51-73, comprising:
not more than 2.5 μg iridium per gram of heparin,
not less than 0.01 and not more than 2.5 μg iridium per gram of heparin,
not less than 0.01 and not more than 1.5 μg iridium per gram of heparin,
not less than 0.01 and not more than 0.5 μg iridium per gram of heparin, or
not less than 0.001 and not more than 0.5 μg iridium per gram of heparin.

75. The composition of any one of claims 51-74, comprising:
not more than 2.5 μg osmium per gram of heparin,
not less than 0.01 and not more than 2.5 μg osmium per gram of heparin,
not less than 0.01 and not more than 1.5 μg osmium per gram of heparin,
not less than 0.01 and not more than 0.5 μg osmium per gram of heparin, or
not less than 0.001 and not more than 0.5 μg osmium per gram of heparin.

76. The composition of any one of claims 51-75, comprising:
not more than 2.5 μg rhodium per gram of heparin,
not less than 0.01 and not more than 2.5 μg rhodium per gram of heparin,
not less than 0.01 and not more than 1.5 μg rhodium per gram of heparin,
not less than 0.01 and not more than 0.5 μg rhodium per gram of heparin, or
not less than 0.001 and not more than 0.5 μg rhodium per gram of heparin.

77. The composition of any one of claims 51-76, comprising:
not more than 2.5 μg ruthenium per gram of heparin,
not less than 0.01 and not more than 2.5 μg ruthenium per gram of heparin,
not less than 0.01 and not more than 1.5 μg ruthenium per gram of heparin,
not less than 0.01 and not more than 0.5 μg ruthenium per gram of heparin, or
not less than 0.001 and not more than 0.5 μg ruthenium per gram of heparin.

78. The composition of any one of claims 51-77, comprising:
not more than 17.5 μg silver per gram of heparin,
not less than 0.1 and not more than 17.5 μg silver per gram of heparin,
not less than 0.1 and not more than 10 μg silver per gram of heparin,
not less than 0.01 and not more than 5 μg silver per gram of heparin, or
not less than 0.001 and not more than 0.5 μg silver per gram of heparin.

79. The composition of any one of claims 51-78, comprising:
not more than 2.5 μg platinum per gram of heparin,
not less than 0.01 and not more than 2.5 μg platinum per gram of heparin,
not less than 0.01 and not more than 1.5 μg platinum per gram of heparin,
not less than 0.01 and not more than 0.5 μg platinum per gram of heparin, or
not less than 0.001 and not more than 0.5 μg platinum per gram of heparin.

80. The composition of any one of claims 51-79, comprising:
not more than 17.5 μg lithium per gram of heparin,
not less than 0.1 and not more than 17.5 μg lithium per gram of heparin,
not less than 0.1 and not more than 10 μg lithium per gram of heparin,
not less than 0.01 and not more than 5 μg lithium per gram of heparin, or
not less than 0.001 and not more than 0.5 μg lithium per gram of heparin.

81. The composition of any one of claims 51-80, comprising:
not more than 50 μg antimony per gram of heparin,
not less than 1 and not more than 50 μg antimony per gram of heparin,
not less than 0.1 and not more than 25 μg antimony per gram of heparin,
not less than 0.1 and not more than 10 μg antimony per gram of heparin, or
not less than 0.001 and not more than 0.5 μg antimony per gram of heparin.

82. The composition of any one of claims 51-81, comprising:
not more than 750 μg barium per gram of heparin,
not less than 10 and not more than 750 μg barium per gram of heparin,
not less than 10 and not more than 500 μg barium per gram of heparin,
not less than 10 and not more than 250 μg barium per gram of heparin, or
not less than 0.001 and not more than 0.5 μg barium per gram of heparin.

83. The composition of any one of claims 51-82, comprising:
not more than 75 μg copper per gram of heparin,
not less than 1 and not more than 75 μg copper per gram of heparin,
not less than 0.1 and not more than 50 μg copper per gram of heparin,
not less than 0.1 and not more than 25 μg copper per gram of heparin, or
not less than 0.001 and not more than 0.5 μg copper per gram of heparin.

84. The composition of any one of claims 51-83, comprising:
not more than 150 μg tin per gram of heparin,
not less than 1 and not more than 150 μg tin per gram of heparin,
not less than 0.1 and not more than 75 μg tin per gram of heparin,
not less than 0.1 and not more than 50 μg tin per gram of heparin, or
not less than 0.001 and not more than 0.5 μg tin per gram of heparin.

85. The composition of any one of claims 51-84, wherein the composition comprises an average droplet diameter of 1 μm to 12 μm.

86. The composition of any one of claims 51-84, wherein the composition comprises an average droplet diameter of 1 μm to 10 μm.

87. The composition of any one of claims 51-84, wherein the composition comprises an average droplet diameter of 1 μm to 8 μm.

88. The composition of any one of claims 51-84, wherein the composition comprises an average droplet diameter of 1 μm to 5 μm.

89. The composition of any one of claims 51-84, wherein the composition comprises an average droplet diameter of 2 μm to 4 μm.

90. The composition of any one of claims 51-84, wherein the composition comprises an average droplet diameter of 2 μm to 3 μm.
